SEQUENCE LISTING (146 sequences)

SEQ ID NO: 1 - protein, 286 aa, Artificial Sequence
  amino acids 266 to 551 of interleukin-2 receptor beta  (IL2RB), NP_000869.1 (IL2Rbeta endodomain)
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 2 - protein, 195 aa, Artificial Sequence
  amino acids 265 to 459 of interleukin-7 receptor-alpha (IL7R-alpha), NP_002176.2
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 3 - protein, 230 aa, Artificial Sequence
  amino acids 292 to 521 of interleukin-9 receptor (IL9R), NP_002177.2
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 4 - protein, 569 aa, Artificial Sequence
  amino acids 257 to 825 of interleukin-4 receptor-alpha (IL4R-alpha), NPJD00409.1
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 5 - protein, 437 aa, Artificial Sequence
  amino acids 461 to 897 of IL3RB, NP_000386.1
RFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 6 - protein, 189 aa, Artificial Sequence
  amino acids 314 to 502 of IL17RB, NP_061195.2
RHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 7 - protein, 91 aa, Artificial Sequence
  IL7R-alpha 2Y truncated
KKRIKPIVWPSLPDHKKTLEHLCKKPRKNLNVSFNPESFLDCQIHRVDDIQARDEVEGFLQDTFPQQPILTSLGSNQEEAYVTMSSFYQNQ

SEQ ID NO: 8 - protein, 4 aa, Artificial Sequence
  STAT5 association motif
  misc_feature (2)..(3): Xaa can be any naturally occurring amino acid
  MISC_FEATURE (4)..(4): Xaa may be Phe or Leu
YXXX

SEQ ID NO: 9 - protein, 4 aa, Artificial Sequence
  STAT5 association motif
YCTF

SEQ ID NO: 10 - protein, 4 aa, Artificial Sequence
  STAT5 association motif
YFFF

SEQ ID NO: 11 - protein, 4 aa, Artificial Sequence
  STAT5 association motif
YLSL

SEQ ID NO: 12 - protein, 5 aa, Artificial Sequence
  STAT5 association motif
YLSLQ

SEQ ID NO: 13 - protein, 57 aa, Artificial Sequence
  JAK1-binding motif
KVLKCNTPDPSKFFSQLSSEHGGDVQKWLSSPFPSSSFSPGGLAPEISPLEVLERDK

SEQ ID NO: 14 - protein, 45 aa, Artificial Sequence
  JAK1-binding motif
NPWFQRAKMPRALDFSGHTHPVATFQPSRPESVNDLFLCPQKELT

SEQ ID NO: 15 - protein, 42 aa, Artificial Sequence
  JAK1-binding motif
GYICLRNSLPKVLNFHNFLAWPFPNLPPLEAMDMVEVIYINR

SEQ ID NO: 16 - protein, 42 aa, Artificial Sequence
  JAK1-binding motif
PLKEKSIILPKSLISVVRSATLETKPESKYVSLITSYQPFSL

SEQ ID NO: 17 - protein, 43 aa, Artificial Sequence
  JAK1-binding motif
RRRKKLPSVLLFKKPSPFIFISQRPSPETQDTIHPLDEEAFLK

SEQ ID NO: 18 - protein, 45 aa, Artificial Sequence
  JAK1-binding motif
YIHVGKEKHPANLILIYGNEFDKRFFVPAEKIVINFITLNISDDS

SEQ ID NO: 19 - protein, 41 aa, Artificial Sequence
  JAK1-binding motif
RYVTKPPAPPNSLNVQRVLTFQPLRFIQEHVLIPVFDLSGP

SEQ ID NO: 20 - protein, 43 aa, Artificial Sequence
  JAK2-binding motif
NYVFFPSLKPSSSIDEYFSEQPLKNLLLSTSEEQIEKCFIIEN

SEQ ID NO: 21 - protein, 49 aa, Artificial Sequence
  JAK2-binding motif
YWFHTPPSIPLQIEEYLKDPTQPILEALDKDSSPKDDVWDSVSIISFPE

SEQ ID NO: 22 - protein, 51 aa, Artificial Sequence
  JAK2-binding motif
YAFSPRNSLPQHLKEFLGHPHHNTLLFFSFPLSDENDVFDKLSVIAEDSES

SEQ ID NO: 23 - protein, 94 aa, Artificial Sequence
  truncated IL2Rbeta endodomain - Y510
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 24 - protein, 208 aa, Artificial Sequence
  truncated IL2Rbeta endodomain - Y510 & Y392
NCRNTGPWLKKVLKCNTPDPSKFFSQLSSEHGGDVQKWLSSPFPSSSFSPGGLAPEISPLEVLERDKVTQLLDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 25 - protein, 52 aa, Artificial Sequence
  JAK3-binding motif
ERTMPRIPTLKNLEDLVTEYHGNFSAWSGVSKGLAESLQPDYSERLCLVSEI

SEQ ID NO: 26 - protein, 86 aa, Artificial Sequence
  JAK3-binding motif
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 27 - protein, 113 aa, Artificial Sequence
  intracellular signaling domain of human CD3 zeta chain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 41 aa, Artificial Sequence
  CD28 signaling domain
RSKRSRLLHSDYMNMTPRRPGPTRKHYQPYAPPRDFAAYRS

SEQ ID NO: 29 - protein, 48 aa, Artificial Sequence
  CD27 signaling domain
QRRKYRSNKGESPVEPAEPCHYSCPREEEGSTIPIQEDYRKPEPACSP

SEQ ID NO: 30 - protein, 42 aa, Artificial Sequence
  OX40 signalling domain
ALYLLRRDQRLPPDAHKPPGGGSFRTPIQEEQADAHSTLAKI

SEQ ID NO: 31 - protein, 42 aa, Artificial Sequence
  41BB signalling domain
KRGRKKLLYIFKQPFMRPVQTTQEEDGCSCRFPEEEEGGCEL

SEQ ID NO: 32 - protein, 38 aa, Artificial Sequence
  ICOS signalling domain
CWLTKKKYSSSVHDPNGEYMFMRAVNTAKKSRLTDVTL

SEQ ID NO: 33 - protein, 197 aa, Artificial Sequence
  TNFRSF25 signalling domain
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 34 - protein, 246 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLG

SEQ ID NO: 35 - protein, 27 aa, Artificial Sequence
  transmembrane domain, CD28 transmembrane
FWVLVVVGGVLACYSLLVTVAFIIFWV

SEQ ID NO: 36 - protein, 66 aa, Artificial Sequence
  transmembrane domain, CD28 hinge and transmembrane sequence
IEVMYPPPYLDNEKSNGTIIHVKGKHLCPSPLFPGPSKPFWVLVVVGGVLACYSLLVTVAFIIFWV

SEQ ID NO: 37 - protein, 68 aa, Artificial Sequence
  transmembrane and intracellular portion of human CD28
FWVLVVVGGVLACYSLLVTVAFIIFWVRSKRSRLLHSDYMNMTPRRPGPTRKHYQPYAPPRDFAAYRS

SEQ ID NO: 38 - protein, 27 aa, Artificial Sequence
  transmembrane domain, CD28
FWVLVVVGGVLACYSLLVTVAFIIFWV

SEQ ID NO: 39 - protein, 10 aa, Artificial Sequence
  c-Myc tag
EQKLISEEDL

SEQ ID NO: 40 - protein, 70 aa, Artificial Sequence
  CD28 hinge/transmembrane domain with integrated c-Myc tag
IEVEQKLISEEDLLDNEKSNGTIIHVKGKHLCPSPLFPGPSKPFWVLVVVGGVLACYSLLVTVAFIIFWV

SEQ ID NO: 41 - protein, 21 aa, Artificial Sequence
  leader sequence
MALPVTALLLPLALLLHAARP

SEQ ID NO: 42 - protein, 834 aa, Artificial Sequence
  chimeric antigen receptor (CAR) containing HLA-A2 scFV, c-Myc tag, CD28, IL2RB-Y510, CD3 zeta endodomain
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLGAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRETRGGGATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 43 - protein, 639 aa, Artificial Sequence
  CAR containing HLA-A2 scFV, c-Myc tag, CD28, IL2RG-T52, IL2RB-Y510, CD3 zeta endodomain
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLGAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSERTMPRIPTLKNLEDLVTEYHGNFSAWSGVSKGLAESLQPDYSERLCLVSEINCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 657 aa, Artificial Sequence
  CAR containing HLA-A2 scFV, c-Myc tag, CD28, IL2RG-T52, IL7RA-2Y, CD3 zeta endodomain
MALPVTALLLPLALLLHAARPQVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLGAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSERTMPRIPTLKNLEDLVTEYHGNFSAWSGVSKGLAESLQPDYSERLCLVSEIKKRIKPIVWPSLPDHKKTLEHLCKKPRKNLNVSFNPESFLDCQIHRVDDIQARDEVEGFLQDTFPQQPILTSLGSNQEEAYVTMSSFYQNQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 45 - protein, 299 aa, Artificial Sequence
  illustrative endodomain sequence comprising CD28, IL2RG-T52, IL2RB-Y510, CD3 zeta signalling domains
RSKRSRLLHSDYMNMTPRRPGPTRKHYQPYAPPRDFAAYRSERTMPRIPTLKNLEDLVTEYHGNFSAWSGVSKGLAESLQPDYSERLCLVSEINCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 22 aa, Artificial Sequence
  P2A peptide - cleavage domain
GSGATNFSLLKQAGDVEENPGP

SEQ ID NO: 47 - protein, 21 aa, Artificial Sequence
  T2A peptide - cleavage domain
GSGEGRGSLLTCGDVEENPGP

SEQ ID NO: 48 - protein, 23 aa, Artificial Sequence
  E2A peptide - cleavage domain
GSGQCTNYALLKLAGDVESNPGP

SEQ ID NO: 49 - protein, 25 aa, Artificial Sequence
  F2A peptide - cleavage domain
GSGVKQTLNFDLLKLAGDVESNPGP

SEQ ID NO: 50 - protein, 4 aa, Artificial Sequence
  Furin site - cleavage domain
  misc_feature (2)..(3): Xaa can be any naturally occurring amino acid
RXXR

SEQ ID NO: 51 - protein, 4 aa, Artificial Sequence
  Furin site - cleavage domain
RRKR

SEQ ID NO: 52 - protein, 4 aa, Artificial Sequence
  STAT3 association motif
  misc_feature (2)..(3): Xaa can be any naturally occurring amino acid
YXXQ

SEQ ID NO: 53 - protein, 317 aa, Artificial Sequence
  illustrative endodomain sequence comprising CD28, IL2RG-T52, IL7RA-2Y, CD3 zeta signalling domains
RSKRSRLLHSDYMNMTPRRPGPTRKHYQPYAPPRDFAAYRSERTMPRIPTLKNLEDLVTEYHGNFSAWSGVSKGLAESLQPDYSERLCLVSEIKKRIKPIVWPSLPDHKKTLEHLCKKPRKNLNVSFNPESFLDCQIHRVDDIQARDEVEGFLQDTFPQQPILTSLGSNQEEAYVTMSSFYQNQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 54 - protein, 120 aa, Artificial Sequence
  variable heavy domain
VQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTV

SEQ ID NO: 55 - protein, 117 aa, Artificial Sequence
  variable heavy domain
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGT

SEQ ID NO: 56 - protein, 117 aa, Artificial Sequence
  variable heavy domain
QVQLVQSGGGVVQPGGSMRVSCAASGVTLSDYGMHWVRQAPGKGLEWVAFIRNDGSDKYYADSVRGRFTISRDNSKKTVFLQMNSLRAEDTAVYYCAKNGESGPLDYWYFDLWGRGT

SEQ ID NO: 57 - protein, 117 aa, Artificial Sequence
  variable heavy domain
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGT

SEQ ID NO: 58 - protein, 108 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLG

SEQ ID NO: 59 - protein, 107 aa, Artificial Sequence
  variable light domain
DVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 60 - protein, 107 aa, Artificial Sequence
  variable light domain
DIVLMQSPSFLSASVGDRVTITCRASHGINNYLAWYQQKPGKAPKLLIYAASTLQSGVPSRFSGSGSGTEFTLTISSLQPEDFATYYCQQYDSYPPTFGRTKVEIKR

SEQ ID NO: 61 - protein, 107 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDFATYYCQQYSSFPLTFGGGTKVDIK

SEQ ID NO: 62 - protein, 107 aa, Artificial Sequence
  variable light domain
DVVMTQSPSSLSASVGDRVTITCQASQDISNYLNWYQQEPGKAPKLLIYDETHLDSGVPSRFTGSRSGTDFTLTISSLQPEDFATYYCQQYDSLPPTFGGGTKVDIK

SEQ ID NO: 63 - protein, 107 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDIATYYCQQYDNLPITFGGGTKVDIK

SEQ ID NO: 64 - protein, 107 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDIATYYCQQYDNLPSTFGGGTKVDIK

SEQ ID NO: 65 - protein, 107 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDFGTYYCQQYNTYPLTFGGGTKVDIK

SEQ ID NO: 66 - protein, 107 aa, Artificial Sequence
  variable light domain
DVVMTQSPSSLTASVGDRVTITCQASQDISNYLNWYQQKPGKAPKLLIYDASNLETGVPSRFSGSGSGTDFTLSIDSLQPEDFATYYCQQYHTYPLTFGGGTKVDIK

SEQ ID NO: 67 - protein, 107 aa, Artificial Sequence
  variable light domain
DVVMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVDIK

SEQ ID NO: 68 - protein, 107 aa, Artificial Sequence
  variable light domain
DVVMTQSPSSLSASVGDRVTITCRTSQGISSALAWYQQKPGKAPKLLIYDASSLESGVPSRFSGSGSGTDFTLTISSLQPEDFATYYCQQFNNYPLTFGGGTKVDIK

SEQ ID NO: 69 - protein, 107 aa, Artificial Sequence
  variable light domain
DVVMTQSPSSLSASVGDRVTITCQASQDISNYLAWYQQKPGRAPTLLIFAASNLQSGVPSRFSGSGSGTEFTLTISGLQPEDFATYYCLQDSSYPPTFGGGTKVDIK

SEQ ID NO: 70 - protein, 107 aa, Artificial Sequence
  variable light domain
DVVMTQSPSTLSASVGDRVTITCRASQSISSWLAWYQQKPGRAPTLLIYKASNLQSGVPSRFSGSGSGTEFTLTISSLQPDDFASYYCQQYSNYPLTFGGGTKVDIK

SEQ ID NO: 71 - protein, 107 aa, Artificial Sequence
  variable light domain
DVVMTQSPSFLSASVGDRVTITCRASHGISNYFAWYQQKPGKAPKLLIYATSTLQSGVPSRFSGSGSGTEFTLTISGLQPEDFATYYCQQYSSYPLTFGGGTKVDIK

SEQ ID NO: 72 - protein, 106 aa, Artificial Sequence
  variable light domain
DVVMTQSPSTLSAYVGDRITITCRASRGSNYLAWYQQKPGKAPKLLIYATSTLQSGVPLRFSGSGSGTEFTLTISGLQPEDFATYYCQQYDSYPPTFGGGTKVDIK

SEQ ID NO: 73 - protein, 238 aa, Artificial Sequence
  antigen binding domain sequence which is capable of binding to HLA-A2
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGSSGGGGSGGGGSGGGGSDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 74 - protein, 241 aa, Artificial Sequence
  antigen binding domain sequence which is capable of binding to HLA-A2
QVQLVQSGGGVVQPGGSMRVSCAASGVTLSDYGMHWVRQAPGKGLEWVAFIRNDGSDKYYADSVRGRFTISRDNSKKTVFLQMNSLRAEDTAVYYCAKNGESGPLDYWYFDLWGRGTSSGGGGSGGGGSGGGGSDIVLMQSPSFLSASVGDRVTITCRASHGINNYLAWYQQKPGKAPKLLIYAASTLQSGVPSRFSGSGSGTEFTLTISSLQPEDFATYYCQQYDSYPPTFGRTKVEIKR

SEQ ID NO: 75 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDFATYYCQQYSSFPLTFGGGTKVDIK

SEQ ID NO: 76 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQEPGKAPKLLIYDETHLDSGVPSRFTGSRSGTDFTLTISSLQPEDFATYYCQQYDSLPPTFGGGTKVDIK

SEQ ID NO: 77 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPITFGGGTKVDIK

SEQ ID NO: 78 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPSTFGGGTKVDIK

SEQ ID NO: 79 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDFGTYYCQQYNTYPLTFGGGTKVDIK

SEQ ID NO: 80 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLTASVGDRVTITCQASQDISNYLNWYQQKPGKAPKLLIYDASNLETGVPSRFSGSGSGTDFTLSIDSLQPEDFATYYCQQYHTYPLTFGGGTKVDIK

SEQ ID NO: 81 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVDIK

SEQ ID NO: 82 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCRTSQGISSALAWYQQKPGKAPKLLIYDASSLESGVPSRFSGSGSGTDFTLTISSLQPEDFATYYCQQFNNYPLTFGGGTKVDIK

SEQ ID NO: 83 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSSLSASVGDRVTITCQASQDISNYLAWYQQKPGRAPTLLIFAASNLQSGVPSRFSGSGSGTEFTLTISGLQPEDFATYYCLQDSSYPPTFGGGTKVDIK

SEQ ID NO: 84 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSTLSASVGDRVTITCRASQSISSWLAWYQQKPGRAPTLLIYKASNLQSGVPSRFSGSGSGTEFTLTISSLQPDDFASYYCQQYSNYPLTFGGGTKVDIK

SEQ ID NO: 85 - protein, 241 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSFLSASVGDRVTITCRASHGISNYFAWYQQKPGKAPKLLIYATSTLQSGVPSRFSGSGSGTEFTLTISGLQPEDFATYYCQQYSSYPLTFGGGTKVDIK

SEQ ID NO: 86 - protein, 240 aa, Artificial Sequence
  antigen binding domain sequence which is capable of binding to HLA-A2
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTSSGGGGSGGGGSGGGGSDVVMTQSPSTLSAYVGDRITITCRASRGSNYLAWYQQKPGKAPKLLIYATSTLQSGVPLRFSGSGSGTEFTLTISGLQPEDFATYYCQQYDSYPPTFGGGTKVDIK

SEQ ID NO: 87 - protein, 21 aa, Artificial Sequence
  transmembrane domain, CD8alpha transmembrane
IYIWAPLAGTCGVLLLSLVIT

SEQ ID NO: 88 - protein, 72 aa, Artificial Sequence
  CD8alpha hinge domain and CD28 transmembrane domain
TTTPAPRPPTPAPTIASQPLSLRPEACRPAAGGAVHTRGLDFACDFWVLVVVGGVLACYSLLVTVAFIIFWV

SEQ ID NO: 89 - protein, 60 aa, Artificial Sequence
  CD28 hinge domain and CD8alpha transmembrane domain
IEVMYPPPYLDNEKSNGTIIHVKGKHLCPSPLFPGPSKPIYIWAPLAGTCGVLLLSLVIT

SEQ ID NO: 90 - protein, 5 aa, Artificial Sequence
  GL heavy chain variable region (VH) complementarity determining region (CDR), GL VH CDR1
DYGMH

SEQ ID NO: 91 - protein, 17 aa, Artificial Sequence
  GL VH CDR2
FIRNDGSDKYYADSVKG

SEQ ID NO: 92 - protein, 14 aa, Artificial Sequence
  GL VH CDR3
NGESGPLDYWYFDL

SEQ ID NO: 93 - protein, 5 aa, Artificial Sequence
  3PB2 VH CDR1
DYGMH

SEQ ID NO: 94 - protein, 17 aa, Artificial Sequence
  3PB2 VH CDR2
FIRNDGSDKYYADSVKG

SEQ ID NO: 95 - protein, 14 aa, Artificial Sequence
  3PB2 VH CDR3
NGESGPLDYWYLDL

SEQ ID NO: 96 - protein, 5 aa, Artificial Sequence
  3PC4 VH CDR1
DYGMH

SEQ ID NO: 97 - protein, 17 aa, Artificial Sequence
  3PC4 VH CDR2
FIRNDGSDKYYADSVRG

SEQ ID NO: 98 - protein, 14 aa, Artificial Sequence
  3PC4 VH CDR3
NGESGPLDYWYFDL

SEQ ID NO: 99 - protein, 5 aa, Artificial Sequence
  3PF12 VH CDR1
DYGMH

SEQ ID NO: 100 - protein, 17 aa, Artificial Sequence
  3PF12 VH CDR2
FIRNDGSDKYYADSVKG

SEQ ID NO: 101 - protein, 14 aa, Artificial Sequence
  3PF12 VH CDR3
NGESGPLDYWYFDL

SEQ ID NO: 102 - protein, 11 aa, Artificial Sequence
  GL light chain variable region (VL) CDR1
QASQDISNYLN

SEQ ID NO: 103 - protein, 7 aa, Artificial Sequence
  GL VL CDR2
DASNLET

SEQ ID NO: 104 - protein, 9 aa, Artificial Sequence
  GL VL CDR3
QQYDNLPPT

SEQ ID NO: 105 - protein, 11 aa, Artificial Sequence
  3PB2 VL CDR1
QSSLDISHYLN

SEQ ID NO: 106 - protein, 7 aa, Artificial Sequence
  3PB2 VL CDR2
DASNLET

SEQ ID NO: 107 - protein, 9 aa, Artificial Sequence
  3PB2 VL CDR3
QQYDNLPLT

SEQ ID NO: 108 - protein, 11 aa, Artificial Sequence
  3PC4 VL CDR1
RASHGINNYLA

SEQ ID NO: 109 - protein, 7 aa, Artificial Sequence
  3PC4 VL CDR2
AASTLQS

SEQ ID NO: 110 - protein, 9 aa, Artificial Sequence
  3PC4 VL CDR3
QQYDSYPPT

SEQ ID NO: 111 - protein, 11 aa, Artificial Sequence
  3PF12 VL CDR1
QASQDISNYLN

SEQ ID NO: 112 - protein, 7 aa, Artificial Sequence
  3PF12 VL CDR2
DASNLET

SEQ ID NO: 113 - protein, 9 aa, Artificial Sequence
  3PF12 VL CDR3
QQYSSFPLT

SEQ ID NO: 114 - protein, 11 aa, Artificial Sequence
  C12 VL CDR1
QASQDISNYLN

SEQ ID NO: 115 - protein, 7 aa, Artificial Sequence
  C12 VL CDR2
DETHLDS

SEQ ID NO: 116 - protein, 9 aa, Artificial Sequence
  C12 VL CDR3
QQYDSLPPT

SEQ ID NO: 117 - protein, 11 aa, Artificial Sequence
  E7 VL CDR1
QASQDISNYLN

SEQ ID NO: 118 - protein, 7 aa, Artificial Sequence
  E7 VL CDR2
DASNLET

SEQ ID NO: 119 - protein, 9 aa, Artificial Sequence
  E7 VL CDR3
QQYDNLPIT

SEQ ID NO: 120 - protein, 11 aa, Artificial Sequence
  H10 VL CDR1
QASQDISNYLN

SEQ ID NO: 121 - protein, 7 aa, Artificial Sequence
  H10 VL CDR2
DASNLET

SEQ ID NO: 122 - protein, 9 aa, Artificial Sequence
  H10 VL CDR3
QQYDNLPST

SEQ ID NO: 123 - protein, 11 aa, Artificial Sequence
  B8 VL CDR1
QASQDISNYLN

SEQ ID NO: 124 - protein, 7 aa, Artificial Sequence
  B8 VL CDR2
DASNLET

SEQ ID NO: 125 - protein, 9 aa, Artificial Sequence
  B8 VL CDR3
QQYNTYPLT

SEQ ID NO: 126 - protein, 11 aa, Artificial Sequence
  D2 VL CDR1
QASQDISNYLN

SEQ ID NO: 127 - protein, 7 aa, Artificial Sequence
  D2 VL CDR2
DASNLET

SEQ ID NO: 128 - protein, 9 aa, Artificial Sequence
  D2 VL CDR3
QQYHTYPLT

SEQ ID NO: 129 - protein, 11 aa, Artificial Sequence
  B10 VL CDR1
QASQDISNYLN

SEQ ID NO: 130 - protein, 7 aa, Artificial Sequence
  B10 VL CDR2
DASNLET

SEQ ID NO: 131 - protein, 9 aa, Artificial Sequence
  B10 VL CDR3
QQYDNLPLT

SEQ ID NO: 132 - protein, 11 aa, Artificial Sequence
  2A9 VL CDR1
RTSQGISSALA

SEQ ID NO: 133 - protein, 7 aa, Artificial Sequence
  2A9 VL CDR2
DASSLES

SEQ ID NO: 134 - protein, 9 aa, Artificial Sequence
  2A9 VL CDR3
QQFNNYPLT

SEQ ID NO: 135 - protein, 11 aa, Artificial Sequence
  3B12 VL CDR1
QASQDISNYLA

SEQ ID NO: 136 - protein, 7 aa, Artificial Sequence
  3B12 VL CDR2
AASNLQS

SEQ ID NO: 137 - protein, 9 aa, Artificial Sequence
  3B12 VL CDR3
LQDSSYPPT

SEQ ID NO: 138 - protein, 11 aa, Artificial Sequence
  2D4 VL CDR1
RASQSISSWLA

SEQ ID NO: 139 - protein, 7 aa, Artificial Sequence
  2D4 VL CDR2
KASNLQS

SEQ ID NO: 140 - protein, 9 aa, Artificial Sequence
  2D4 VL CDR3
QQYSNYPLT

SEQ ID NO: 141 - protein, 11 aa, Artificial Sequence
  3D4 VL CDR1
RASHGISNYFA

SEQ ID NO: 142 - protein, 7 aa, Artificial Sequence
  3D4 VL CDR2
ATSTLQS

SEQ ID NO: 143 - protein, 9 aa, Artificial Sequence
  3D4 VL CDR3
QQYSSYPLT

SEQ ID NO: 144 - protein, 10 aa, Artificial Sequence
  B3 VL CDR1
RASRGSNYLA

SEQ ID NO: 145 - protein, 7 aa, Artificial Sequence
  B3 VL CDR2
ATSTLQS

SEQ ID NO: 146 - protein, 9 aa, Artificial Sequence
  B3 VL CDR3
QQYDSYPPT

(19) United States
(12) Patent Application Publication
(10) Pub. No.: US 2021/0338726 A1
(43) Pub. Date: Nov. 4, 2021

(54) ENGINEERED REGULATORY T CELL

(71) Applicant: KING'S COLLEGE LONDON, London (GB)

(72) Inventors: Marc MARTINEZ-LLORDELLA, London (GB); Alberto SANCHEZ-FUEYO, London (GB); Giovanna LOMBARDI, London (GB)

(21) Appl. No.: 17/272,126

(22) PCT Filed: Aug. 30, 2019
(86) PCT No.: PCT/GB2019/052422

§ 371 Date: Feb. 26, 2021

(30) Foreign Application Priority Data
Aug. 31, 2018 (GB) 1814203.4

(51) Int. Cl. A61K 35/17; C12N 5/0783; A61P 37/06; C07K 14/715; C07K 16/28

(52) U.S. Cl. CPC A61K 35/17; C12N 5/0637; A61P 37/06; C07K 14/7155; A61K 2039/5156; C07K 2319/30; C07K 2319/33; C12N 2510/00; C07K 16/2833

(57) ABSTRACT

The present invention provides an engineered regulatory T cell (Treg) comprising a chimeric antigen receptor (CAR) for use in induction of tolerance to a transplant; treating and/or preventing graft-versus-host disease (GvHD), an autoimmune or allergic disease; to promote tissue repair and/or tissue regeneration; or to ameliorate chronic inflammation secondary to metabolic disorders; wherein the CAR comprises an endodomain which comprises a STAT association motif and a JAK1- and/or a JAK2-binding motif.

### FIELD OF THE INVENTION

The present invention relates to engineered regulatory T cells and therapeutic uses of such cells. In particular, the invention relates to engineered regulatory T cells that are less susceptible to microenvironments with limited IL-2 availability.

### BACKGROUND TO THE INVENTION

Regulatory T cells (Tregs) are immune cells with suppressive function that control cytopathic immune responses and are essential for the maintenance of immunological tolerance. The suppressive properties of Tregs can be exploited therapeutically, for example to improve and/or prevent immune-mediated organ damage in inflammatory disorders, autoimmune diseases and in transplantation. Treg immunotherapies usually involve isolation, culture and expansion of Tregs followed by infusion into patients. As part of this process, Tregs may be incubated with cytokines, drugs, other cells or antigens in order to improve their viability and function and/or to confer them enhanced reactivity against specific antigens. These same objectives can be achieved by genetically engineering Tregs to target a predetermined antigen, for example via a chimeric antigen receptor (CAR).

The growth factor interleukin-2 (IL-2) is essential for the homeostasis of Tregs (generation, proliferation, survival), as well as for their suppressive function and phenotypic stability. Activated conventional T cells (Tcons) are the main source of IL-2 in vivo. Tregs, in contrast, cannot produce IL-2 and depend on paracrine access to IL-2 produced by Tcons present in the microenvironment.

The availability of IL-2 has a critical impact on the therapeutic effects of Tregs expanded in vitro and transferred into patients. This is due to the following: 1) in vitro expansion protocols typically require high concentrations of IL-2, which renders Tregs highly dependent on this cytokine; 2) the concentration of IL-2 is often reduced in patients as a result of the administration of immunosuppressive drugs; and 3) within the inflamed tissue microenvironment access to IL-2 is often limited. Liver transplantation constitutes a particularly challenging indication, given that the levels of IL-2 in the inflamed liver are known to be reduced, which is further aggravated by the routine use of calcineurin inhibitors, which substantially decrease the capacity of Tcons to produce IL-2. The administration of low doses exogenous IL-2 restores the Treg dysfunction induced by calcineurin inhibitors and promotes the accumulation of Tregs in the liver. However, a concern with the therapeutic use of low-dose Treg is the risk of simultaneously activating Tcons, which can enhance tissue damage.

WO 2017/218850 describes engineering Tregs which constitutively express STAT5 in order to provide a productive IL-2 signal. However, several challenges can be predicted with this approach. Constitutive STAT5 expression provides a risk that the engineered Tregs may exert non-specific powerful immunosuppression and, due to their high proliferative rate, they may overgrow the endogenous Treg pool and reduce their TCR repertoire, which could result in autoimmunity. Finally, these engineered Tregs may pose risk of transformation, considering that mutations on STAT5 are known to promote T-cell prolymphocytic leukaemia, and that STAT5 is constitutively activated in many cancers.

Accordingly, there remains a need for approaches to produce engineered Tregs which are less susceptible to microenvironments with limited IL-2 availability and approaches to improve the effectiveness of engineered Tregs to proliferate and survive in subjects who have been administered immunosuppressive drugs.

### SUMMARY OF THE INVENTION

The present inventors have developed an engineered regulatory T cell (Treg) which is capable of providing a productive IL-2 signal upon binding of the Treg to a predetermined antigen. Thus, the engineered Tregs of the present invention address the problem associated with the high IL-2 dependence of adoptively transferred Tregs without requiring exogenous IL-2 to be administered and by providing a productive IL-2 signal in an antigen-specific manner.

Thus, in a first aspect the present invention provides an engineered Treg comprising a chimeric antigen receptor (CAR) for use in induction of tolerance to a transplant; treating and/or preventing graft-versus-host disease (GvHD), an autoimmune or allergic disease; to promote tissue repair and/or tissue regeneration; or to ameliorate chronic inflammation secondary to metabolic disorders; wherein the CAR comprises an endodomain which comprises a STAT5 association motif and a JAK1- and/or a JAK2-binding motif.

In another aspect the present invention provides a pharmaceutical composition comprising an engineered Treg according to the first aspect of the invention for use in induction of tolerance to a transplant; treating and/or preventing GvHD, an autoimmune or allergic disease; to promote tissue repair and/or tissue regeneration; or to ameliorate chronic inflammation secondary to metabolic disorders.

The invention further relates to a method of inducing tolerance to a transplant; treating and/or preventing GvHD, an autoimmune or allergic disease; or to promote tissue repair and/or tissue regeneration; or to ameliorate chronic inflammation secondary to metabolic disorders which comprises the step of administering an engineered Treg or a pharmaceutical composition according to the present invention to a subject.

The present invention also provides the use of an engineered Treg according to the present invention in the manufacture of a medicament for inducing tolerance to a transplant; treating and/or preventing cellular and/or humoral transplant rejection; treating and/or preventing GvHD, an autoimmune or allergic disease; or to promote tissue repair and/or tissue regeneration; or to ameliorate chronic inflammation secondary to metabolic disorders.

Suitably, the subject may be a transplant recipient and the invention is directed to induction of tolerance to a transplant (e.g. a transplanted organ). In particular, the subject may be a transplant recipient undergoing immunosuppression therapy.

In another aspect the present invention provides a CAR comprising an endodomain which comprises a STAT5 association motif and a JAK1- and/or a JAK2-binding motif but does not comprise a STAT3 association motif.

Suitably, the CAR endodomain does not comprise the amino acid sequence YXXQ (SEQ ID NO: 52). Suitably, the IL2Rβ portion of the CAR endodomain does not comprise the amino acid sequence YXXQ (SEQ ID NO: 52).

In a further aspect the present invention provides a CAR comprising an endodomain which comprises a STAT5 association motif, a JAK1- and/or a JAK2-binding motif, and a JAK3-binding motif.

The present invention further provides a polynucleotide encoding the CAR of the invention and a vector encoding the CAR of the invention.

In a further aspect the present invention provides an engineered Foxp3+ Treg comprising a CAR of the invention, and the engineered Foxp3+ Treg comprising a CAR of the invention for use in therapy.

The present invention thus provides an engineered Treg comprising a CAR, which CAR provides a STAT5-mediated pro-survival signal to the Treg exclusively upon CAR binding to its cognate antigen. In particular, after antigen recognition, the present CARs cluster and a signal is transmitted to the engineered Treg via the intracellular signaling domain (endodomain) of the CAR. Because the present CAR comprises an endodomain which comprises a STAT5 association motif and a JAK1- and/or a JAK2-binding motif, clustering of the present CAR leads to STAT5 and JAK1 and/or JAK2 recruitment and activation; and thus provides a signal that enhances the function and the survival of the engineered Treg in an antigen-specific manner without being dependent on the availability of IL-2 in the microenvironment.

The engineered Tregs of the present invention may be particularly effective in providing a survival advantage to the engineered CAR-Tregs after antigen recognition compared to the general T cell population of the subject. In particular, in the context of e.g. transplantation where the use of immunosuppressive drugs reduces the availability of IL-2, the STAT5 signalling of the present CAR-Tregs provides additional survival and functional effects on the cells of the invention in an otherwise disadvantageous microenvironment.

### BRIEF DESCRIPTION OF THE FIGURES

FIG. 1—Diagram illustrating CAR constructs of the invention

FIG. 2—Exemplary designs of anti-HLA.A2 IL2R CAR constructs

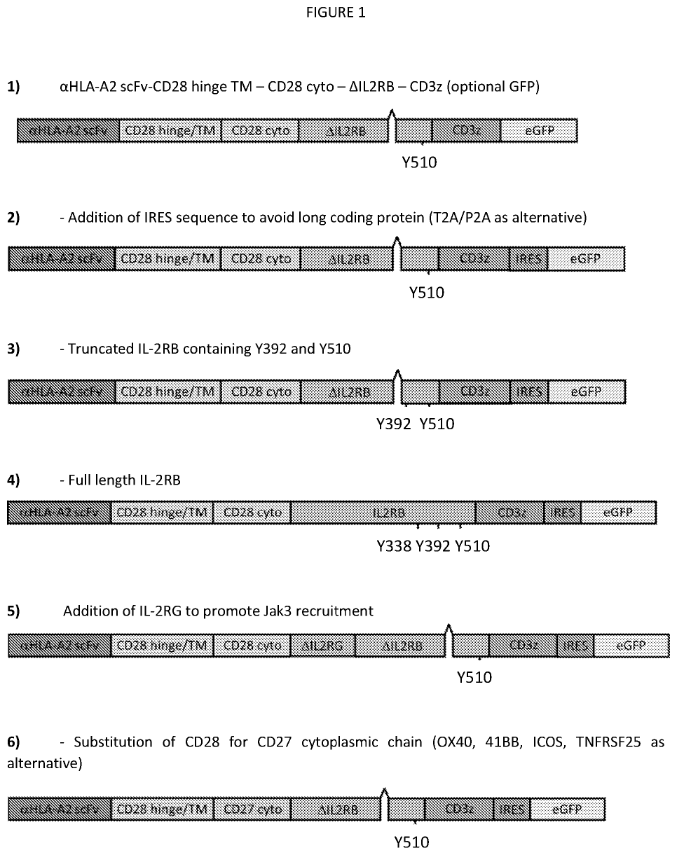

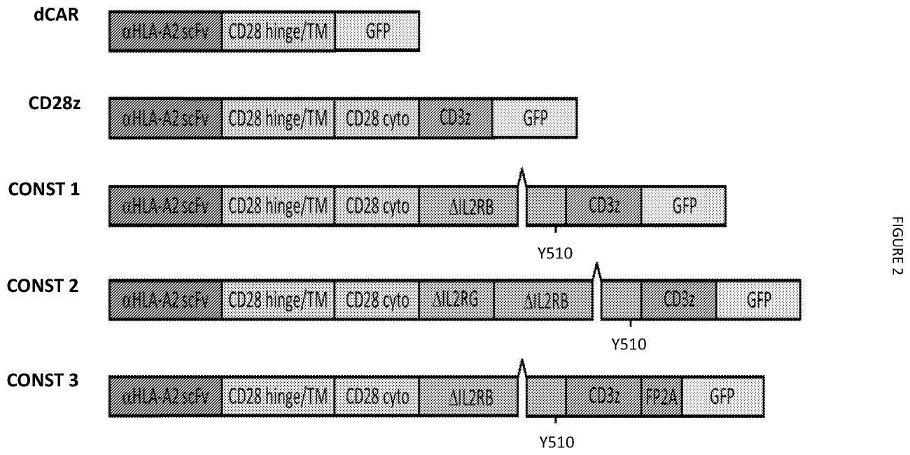

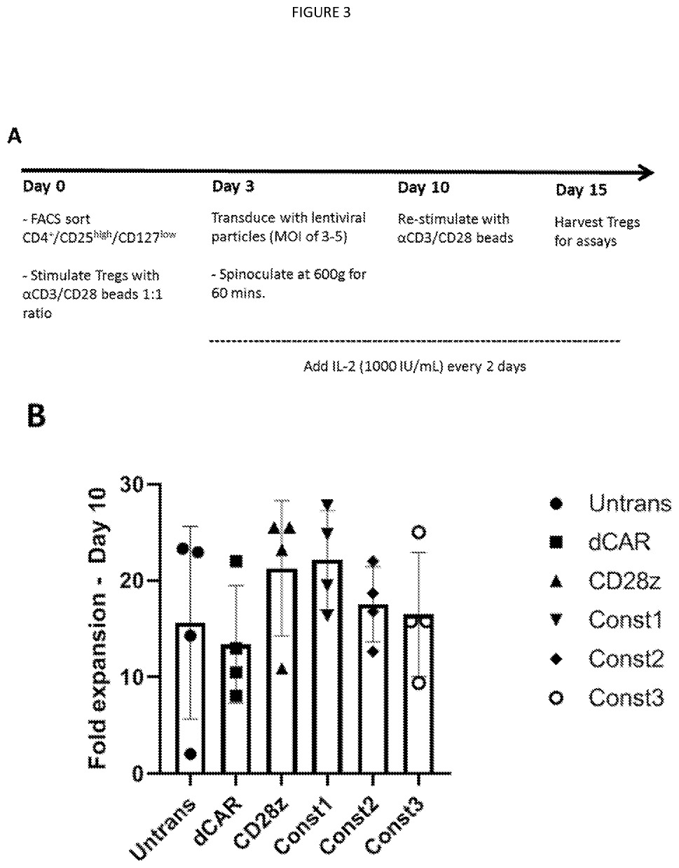

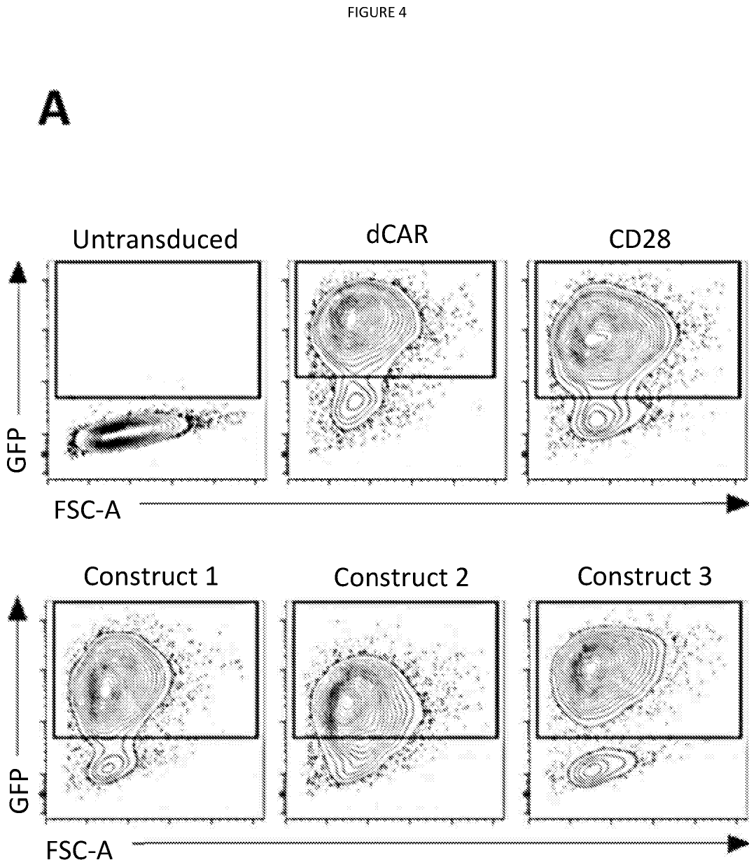

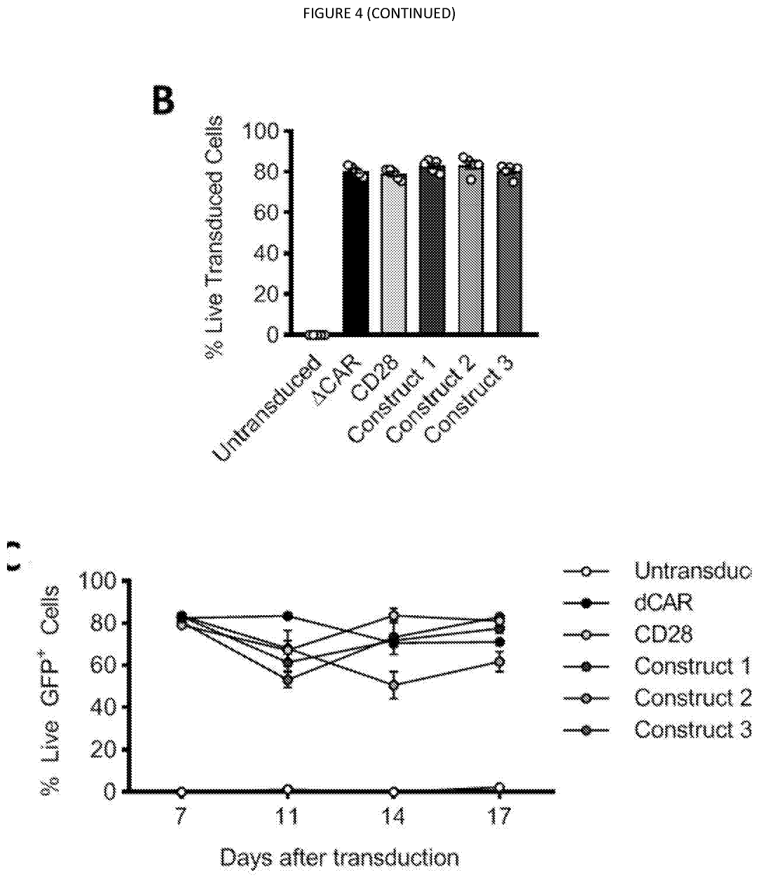

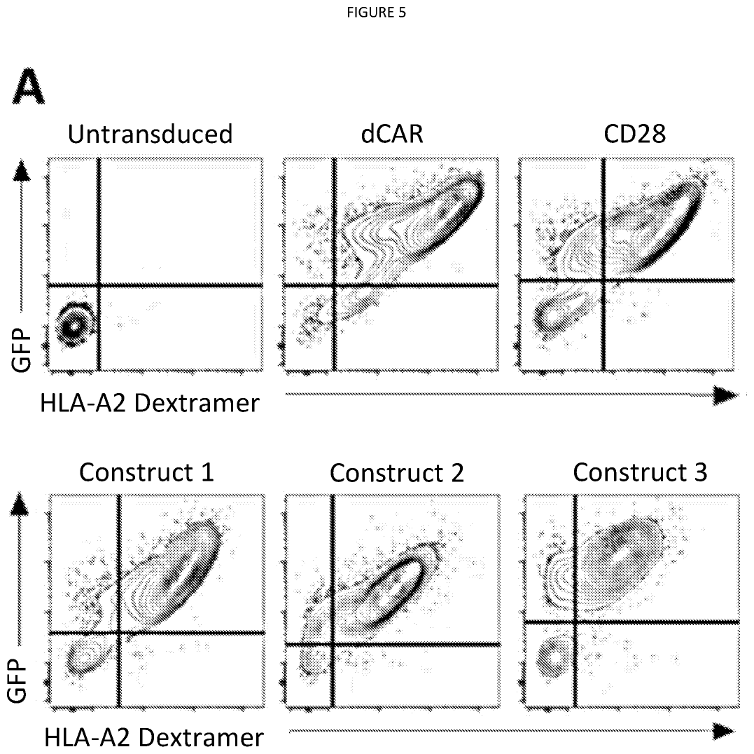

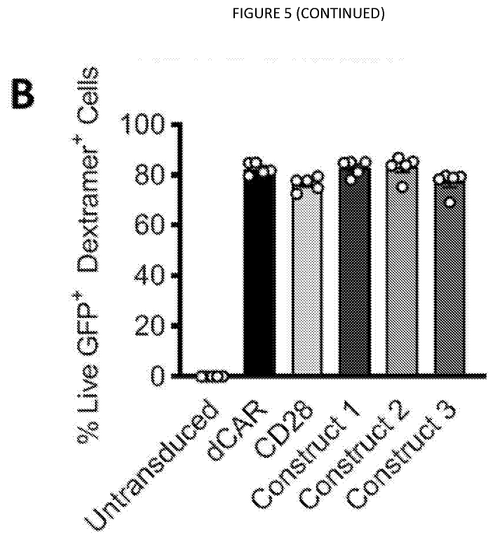

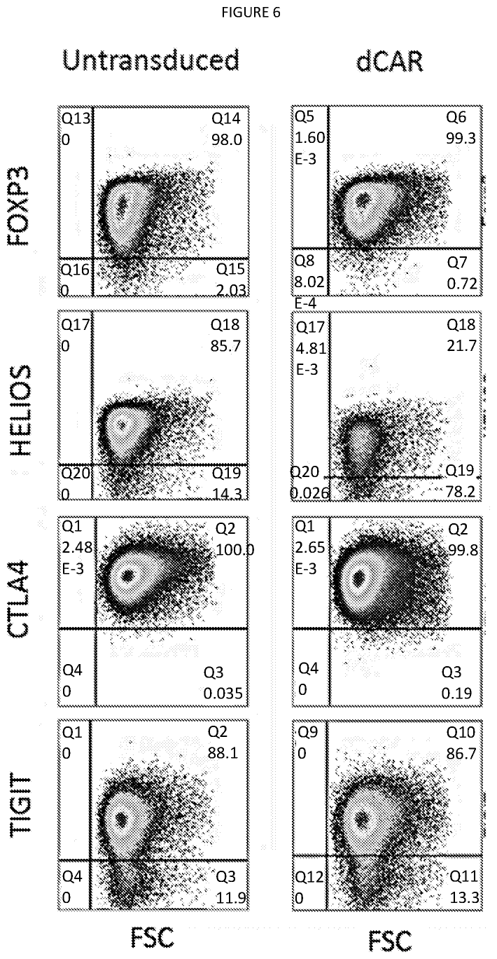

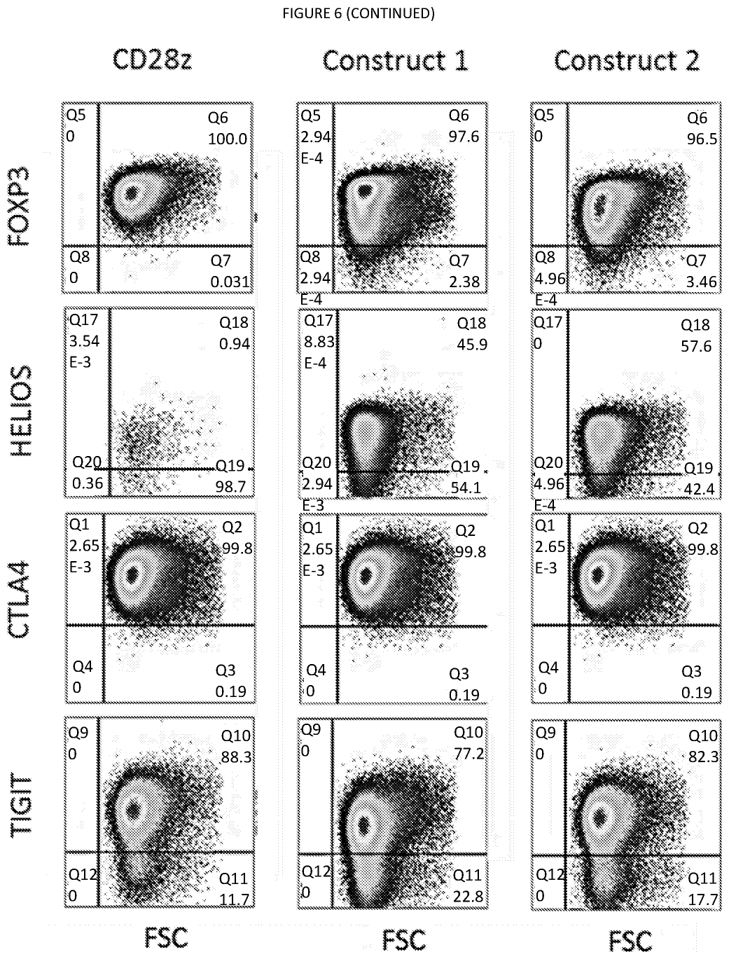

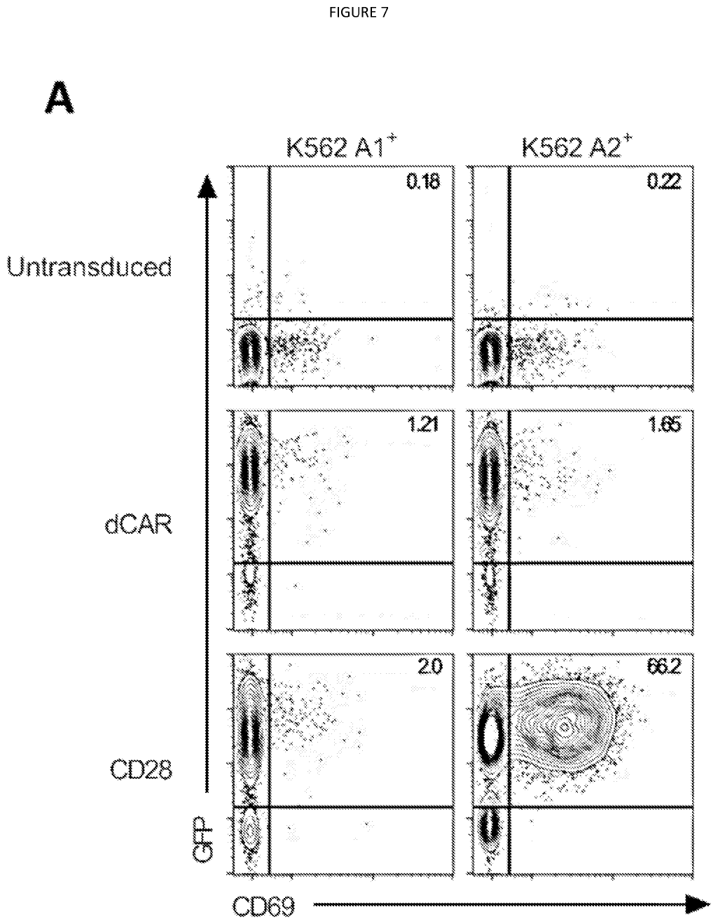

Schematics of exemplary anti-HLA.A2 CAR constructs including different combinations of IL2R endodomain. (A) dCAR construct: HLA.A2 scFv antigen recognition domain; CD28 hinge domain; CD28 TM and eGFP. (B) CD28z construct: HLA.A2 scFv antigen recognition domain; CD28 hinge domain; CD28 TM; CD28 signaling domain; CD3z signaling domain and eGFP. (C) IL2R Construct 1: HLA.A2 scFv antigen recognition domain; CD28 hinge domain; CD28 TM; CD28 signaling domain; truncated IL2RB endodomain; CD3z signaling domain and eGFP. (D) IL2R Construct 1: HLA.A2 scFv antigen recognition domain; CD28 hinge domain; CD28 TM; CD28 signaling domain; truncated IL2RG; truncated IL2RB endodomain; CD3z signaling domain and eGFP. (E) IL2R Construct 1: HLA.A2 scFv antigen recognition domain; CD28 hinge domain; CD28 TM; CD28 signaling domain; truncated IL2RB endodomain; CD3z signaling domain; FP2A cleavage domain and eGFP.

FIG. 3—Generation of anti-HLA.A2 IL2R CAR-Tregs

Schematic illustration showing the generation and expansion of anti-HLA.A2 IL2R CAR-Tregs. (A) Isolated CD4+CD25hiCD127low cells were isolated and activated with anti-CD3/CD28 beads. Three days after activation Tregs were transduced with lentivirus containing the HLA.A2-CAR and the GFP reported gene. Fresh media and 1000 IU/ml IL-2 were added every 2 days. Transduced and untransduced Tregs were cultured during 10 days and GFP was measured to assess transduction efficacy. Tregs were further expanded with fresh anti-CD3/CD28 beads. (B) Fold change expansion of Tregs untransduced or transduced with different CAR constructs on day 10 after activation.

FIG. 4—Quantification of transduction efficacy of anti-HLA.A2 IL2R constructs over time

GFP expression was analysed on Tregs untransduced and transduced with CAR constructs at different time points after cell activation. (A) Representative contour plots of GFP expression from HLA-A2 IL2R CAR Tregs 7 days following transduction. (B) Quantification of GFP⁺ CAR Tregs among live CD4+ cells 7 days following transduction. (C) Quantification of GFP expression from HLA-A2 IL2R CAR Tregs over time.

FIG. 5—Quantification of cell surface expression of anti-HLA.A2 IL2R CAR constructs on transduced Tregs

Membrane expression of CAR construct on untransduced and transduced Tregs was analysed by PE-conjugated HLA-A*0201/CINGVCWTV dextramers (Immudex, Copenhagen, Denmark). (A) Representative contour plots of GFP+Dextramer+ CAR Tregs 7 days following transduction. (B) Quantification of Dextramer+ cells among the GFP+ Tregs on day 7 after transduction.

FIG. 6—Phenotypic characterization of CAR Tregs after polyclonal cell expansion

Tregs were cultured and expanded for 15 days in the presence of anti-CD3/CD28 activation beads and IL-2. Treg related markers FOXP3, HELIOS, CTLA4 and TIGIT were analysed by FACS on untransduced and transduced Tregs to assess phenotypic lineage stability on day 15 of culture.

FIG. 7—Evaluation of the antigen-specificity of anti-HLA.A2 IL2R CAR Tregs

Untransduced and transduced Tregs were cultured for 18 hours in the presence of different stimulus. CD69 and CD137 activation markers were analysed to assess specific and unspecific cell activation. (A) Representative contour plots showing the expression CD69 in response to culture with K562 cells transduced with HLA.A1 or HLA.A2 molecules. GFP signal was used to select the transduced Tregs. (B) Quantification of CD69 and CD137 expression on Tregs 18 hours after culture with media alone (unstimulated), anti-CD3/CD28 beads (unspecific stimulation), K562-HLA.A1 and K562-HLA.A2 cells. (C) Representative histograms showing CD69 expression on Tregs after 18 hours culture with HLA.A1 and HLA.A2 B cell lines. Different cell to cell ratios were used.

FIG. 8—STAT5 phosphorylation analysis as an indicator of IL2R CAR signaling

Transduced CAR Tregs were rested overnight in culture media without IL2. STAT5 phosphorylation of Tregs was assessed by FACS analysis 10 and 120 minutes after culture with media alone, 1000 IU/ml IL-2 or in the presence of HLA.A2-Ig based artificial APCs (produced following the protocol described at DOI: 10.3791/2801). (A) Contour plots showing the expression of GFP and phosphoSTAT5 on transduced CAR-Tregs after 10 minutes culture with media alone, HLA.A2 beads at 1:1 ratio and 1000 IU/ml IL-2. (B) Histograms showing the phosphorylation of STAT5 of Tregs cultured for 120 minutes with HLA.A2 beads 1:1 ratio or media alone (unstim).

FIG. 9—Evaluation of Treg survival after unspecific and HLA.A2 specific activation in the absence of IL-2

CAR transduced Tregs with different constructs were cultured with anti-CD3/28 activation beads and K562.A2 expression cells without the presence of IL-2. Cell survival was assessed 7 days after activation by FACS analysis. (A) Representatives histograms of CAR-Tregs showing cell survival of GFP+ cells based on Viability dye staining on day 7 after activation without IL-2. (B) Percentage of viable cells on GFP+ Tregs after 7 days of culture whit anti-CD3/28 beads and K562-HLA.A2 cells in absence of IL-2 (* p<0.05, ANOVA analysis with Tukey's post hoc correction).

FIG. 10—Treg suppression potency test: Evaluate the immunoregulatory function of Tregs by analysing the modulation of co-stimulatory molecules on B cells

B cell expression of CD80 and CD86 after co-culture with Tregs was analysed to evaluate the capacity of Tregs to reduce the expression of co-stimulatory molecules on antigen presenting cells. Fixed number of alive A2-expressing B cells (20K/well) were co-cultured with titrated numbers of Treg products (A2-negative donors) (200, 100, 50, 25, 12.5K) overnight. FACS analysis of CD86 and CD80 co-stimulatory markers on B cells.

### DETAILED DESCRIPTION OF THE INVENTION

Engineered Regulatory T Cell (Treg)

An “engineered cell” as used herein means a cell which has been modified to comprise or express a polynucleotide which is not naturally encoded by the cell. Methods for engineering cells are known in the art and include, but are not limited to, genetic modification of cells e.g. by transduction such as retroviral or lentiviral transduction, transfection (such as transient transfection—DNA or RNA based) including lipofection, polyethylene glycol, calcium phosphate and electroporation. Any suitable method may be used to introduce a nucleic acid sequence into a cell. Non-viral technologies such as amphipathic cell penetrating peptides may be used to introduce nucleic acid in accordance with the present invention.

Accordingly, the polynucleotide encoding a CAR as described herein is not naturally expressed by a corresponding, unmodified cell. Suitably, an engineered cell is a cell which has been modified e.g. by transduction or by transfection. Suitably, an engineered cell is a cell which has been modified or whose genome has been modified e.g. by transduction or by transfection. Suitably, an engineered cell is a cell which has been modified or whose genome has been modified by retroviral transduction. Suitably, an engineered cell is a cell which has been modified or whose genome has been modified by lentiviral transduction.

As used herein, the term “introduced” refers to methods for inserting foreign DNA or RNA into a cell. As used herein the term introduced includes both transduction and transfection methods. Transfection is the process of introducing nucleic acids into a cell by non-viral methods. Transduction is the process of introducing foreign DNA or RNA into a cell via a viral vector. Engineered cells according to the present invention may be generated by introducing DNA or RNA encoding a CAR as described herein by one of many means including transduction with a viral vector, transfection with DNA or RNA. Cells may be activated and/or expanded prior to, or after, the introduction of a polynucleotide encoding the CAR as described herein, for example by treatment with an anti-CD3 monoclonal antibody or both anti-CD3 and anti-CD28 monoclonal antibodies. The Tregs may also be expanded in the presence of anti-CD3 and anti-CD28 monoclonal antibodies in combination with IL-2. Suitably, IL-2 may be substituted with IL-15. Other components which may be used in a Treg expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

Regulatory T cells (Treg) are immune cells with immunosuppressive function that control cytopathic immune responses and are essential for the maintenance of immunological tolerance.

As used herein, the term Treg refers to a T cell with immunosuppressive function.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tconv) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages.

Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with Tregs of the present invention may proliferate 5%, 10%, 20%, 30%, 40%, 50%, 60%, 70%, 90%, 95% or 99% less than the same Tconv cells cultured in the absence of Tregs of the invention.

Antigen-specific Tconv cells co-cultured with Tregs of the invention may express at least 10%, at least 20%, at least 30%, at least 40%, at least 50%, or at least 60% less effector cytokine than corresponding Tconv cells cultured in the absence of Tregs of the invention.

The effector cytokine may be selected from IL-2, IL-17, TNFα, GM-CSF, IFN-γ, IL-4, IL-5, IL-9, IL-10 and IL-13.

Suitably the effector cytokine may be selected from IL-2, IL-17, TNFα, GM-CSF and IFN-γ.

Suitably, the Treg is a T cell which expresses the markers CD4, CD25 and FOXP3 (CD4⁺CD25⁺FOXP3⁺). “FOXP3” is the abbreviated name of the forkhead box P3 protein. FOXP3 is a member of the FOX protein family of transcription factors and functions as a master regulator of the regulatory pathway in the development and function of regulatory T cells.

Tregs may also express CTLA-4 (cytotoxic T-lymphocyte associated molecule-4) or GITR (glucocorticoid-induced TNF receptor). Treg cells are present in the peripheral blood, lymph nodes, and tissues and include thymus-derived, natural Treg (nTreg) cells and peripherally generated, induced Treg (iTreg) cells.

Suitably, the Treg may be identified using the cell surface markers CD4 and CD25 in the absence of or in combination with low-level expression of the surface protein CD127 (CD4⁺CD25⁺CD127 or CD4⁺CD25⁺CD127^{low}). The use of such markers to identify Tregs is known in the art and described in Liu et al. (JEM; 2006; 203; 7(10); 1701-1711), for example.

The Treg may be a CD4⁺CD25⁺FOXP3⁺ T cell.

The Treg may be a CD4⁺CD25⁺CD127⁻ T cell.

The Treg may be a CD4⁺CD25⁺FOXP3⁺CD127^{-/low} T cell.

The Treg may be natural or thymus-derived, adaptive or peripherally-derived, or in vitro-induced (Abbas, A. K., et al., 2013. Nature immunology, 14(4), p. 307-308).

Suitably, the Treg may be a natural Treg (nTreg). As used herein, the term “natural T reg” means a thymus-derived Treg. Natural T regs are CD4⁺CD25⁺FOXP3⁺ Helios⁺ Neuropilin 1⁺. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

The Treg may have a demethylated Treg-specific demethylated region (TSDR). The TSDR is an important methylation-sensitive element regulating Foxp3 expression (Polansky, J. K., et al., 2008. European journal of immunology, 38(6), pp. 1654-1663).

Further suitable Tregs include, but are not limited to, Tr1 cells (which do not express Foxp3, and have high IL-10 production); CD8⁺FOXP3⁺ T cells; and γδ FOXP3⁺ T cells.

Methods for determining the presence of cell markers are well-known in the art and include, for example, flow cytometry.

Suitably, the cell, such as a Treg, is isolated from peripheral blood mononuclear cells (PBMCs) obtained from a subject. Suitably the subject from whom the PBMCs are obtained is a mammal, preferably a human. Suitably the cell is matched (e.g. HLA matched) or is autologous to the subject to whom the engineered Treg is to be administered. Suitably, the subject to be treated is a mammal, preferably a human. The cell may be generated ex vivo either from a patient's own peripheral blood (1st party), or in the setting of a haematopoietic stem cell transplant from donor peripheral blood (2nd party), or peripheral blood from an unconnected donor (3rd party). Suitably the cell is autologous to the subject to whom the engineered Treg is to be administered.

Suitably, the Treg is isolated from peripheral blood mononuclear cells (PBMCs) obtained from a subject. In a preferred embodiment, the Treg is isolated from peripheral blood mononuclear cells (PBMCs) obtained from a subject and is matched or is autologous to the subject to be treated.

Suitably, the Treg is isolated from the subject to be treated.

Suitably, the Treg is part of a population of Tregs. Suitably, the population of Tregs comprises at least 70% Tregs, such as at least 75, 85, 90, 95, 97, 98 or 99% Tregs. Such a population may be referred to as an “enriched Treg population”.

In some aspects, the Treg may be derived from ex-vivo differentiation of inducible progenitor cells or embryonic progenitor cells to the Treg. A polynucleotide or vector of the invention may be introduced into the inducible progenitor cells or embryonic progenitor cells prior to, or after, differentiation to a Treg.

As used herein, the term “conventional T cell” or Tcon means a T lymphocyte cell which expresses an αβ T cell receptor (TCR) as well as a co-receptor which may be cluster of differentiation 4 (CD4) or cluster of differentiation 8 (CD8) and which does not have an immunosuppressive function. Conventional T cells are present in the peripheral blood, lymph nodes, and tissues. Suitably, the engineered Treg may generated from a Tcon by introducing DNA or RNA coding for FOXP3 in addition to the DNA or RNA coding for the CAR as described herein, by one of many means including transduction with a viral vector, or transfection with DNA or RNA on the same or different vectors. Alternatively, the engineered Treg may be generated from a Tcon by in vitro culture of CD4+CD25-FOXP3-cells in the presence of IL-2 and TGF-β.

Chimeric Antigen Receptor (CAR)

“Chimeric antigen receptor” or “CAR” or “CARs” as used herein refers to engineered receptors which can confer an antigen specificity onto cells (for example Tregs). CARs are also known as artificial T-cell receptors, chimeric T-cell receptors or chimeric immunoreceptors. Preferably the CARs of the invention comprise an extracellular antigen-specific targeting region, a transmembrane domain, optionally one or more co-stimulatory domains, and an intracellular signaling domain (also referred to as an endodomain).

CAR-encoding polynucleotides may be transferred to the Treg using, for example, retroviral vectors. In this way, a large number of antigen-specific T cells can be generated for adoptive cell transfer. When the CAR binds the target-antigen, this results in the transmission of an activating signal to the Treg it is expressed on. Thus the CAR directs the specificity of the engineered Treg towards cells expressing the targeted antigen.

Intracellular Signaling Domain (Endodomain)

The present CAR comprises an endodomain which comprises a STAT5 association motif and a JAK1- and/or a JAK2-binding motif.

“Signal Transducer and Activator of Transcription 5” (STAT5) is a transcription factor involved in the IL-2 signalling pathway that plays a key role in Treg function, stability and survival by promoting the expression of genes such as FOXP3, IL2RA and BCLXL. In order to be functional and translocate into the nucleus, STAT5 needs to be phosphorylated. IL-2 ligation results in STAT5 phosphorylation by activating the Jak1/Jak2 and Jak3 kinases via specific signalling domains present in the IL-2R13 and IL-2Rγ chain, respectively. Although Jak1 (or Jak2) can phosphorylate STAT5 without the need of Jak3, STAT5 activity is increased by the transphosphorylation of both Jak1/Jak2 and Jak3, which stabilizes their activity.

“STAT5 association motif” as used herein refers to an amino acid motif which comprises a tyrosine and is capable of binding a STAT5 polypeptide. Any method known in the art for determining protein:protein interactions may be used to determine whether an association motif is capable of binding to STAT5. For example, co-immunoprecipitation followed by western blot.

Suitably, the CAR endodomain may comprise two or more STAT5 association motifs as defined herein. For example, the CAR endodomain may comprise two, three, four, five or more STAT5 association motifs as defined herein. Preferably, the CAR endodomain may comprise two or three STAT5 association motifs as defined herein.

Suitably, the STAT5 association motif may exist endogenously in a cytoplasmic domain of a transmembrane protein. For example, the STAT5 association motif may be from an interleukin receptor (IL) receptor endodomain or a hormone receptor.

The CAR endodomain may comprise an amino acid sequence selected from any chain of the interleukin receptors where STAT5 is a downstream component, for example, the cytoplasmic domain comprising amino acid numbers 266 to 551 of IL-2 receptor β chain (NCBI REFSEQ: NP_000869.1, SEQ ID NO: 1), amino acid numbers 265 to 459 of IL-7R α chain (NCBI REFSEQ: NP_002176.2, SEQ ID NO: 2), amino acid numbers 292 to 521 of IL-9R chain (NCBI REFSEQ: NP_002177.2, SEQ ID NO: 3), amino acid numbers 257 to 825 of IL-4R α chain (NCBI REFSEQ: NPJD00409.1, SEQ ID NO: 4), amino acid numbers 461 to 897 of IL-3R β chain (NCBI REFSEQ: NP_000386.1, SEQ ID NO: 5) or amino acid numbers 314 to 502 of IL-17R β chain (NCBI REFSEQ: NP_061195.2, SEQ ID NO: 6) may be used. The entire region of the cytoplasmic domain of interleukin receptor chain may be used.

IL7RA (AA 265 to 459 of NP_002176.2)
SEQ ID NO: 2
KKRIKPIVWPSLPDHKKTLEHLCKKPRKNLNVSENPESELDCQIHRVDDI
QARDEVEGFLQDTFPQQLEESEKQRLGGDVQSPNCPSEDVVITPESEGRD
SSLTCLAGNVSACDAPILSSSRSLDCRESGKNGPHVYQDLLLSLGTTNST
LPPPFSLQSGILTLNPVAQGQPILTSLGSNQEEAYVTMSSFYQNQ
IL7RA 2Y truncated:
SEQ ID NO: 7
KKRIKPIVWPSLPDHKKTLEHLCKKPRKNLNVSFNPESFLDCQIHRVDDI
QARDEVEGFLQDTFPQQPILTSLGSNQEEAYVTMSSFYQNQ
IL9R (AA 292 to 521 of NP_002177.2)
SEQ ID NO: 3
KLSPRVKRIFYQNVPSPAMFFQPLYSVHNGNFQTWMGAHGAGVLLSQDCA
GTPQGALEPCVQEATALLTCGPARPWKSVALEEEQEGPGTRLPGNLSSED
VLPAGCTEWRVQTLAYLPQEDWAPTSLTRPAPPDSEGSRSSSSSSSSNNN
NYCALGCYGGWHLSALPGNTQSSGPIPALACGLSCDHQGLETQQGVAWVL
AGHCQRPGLHEDLQGMLLPSVLSKARSWTF
IL4RA (AA 257 to 825 of NPJD00409.1)
SEQ ID NO: 4
KIKKEWWDQIPNPARSRLVAIIIQDAQGSQWEKRSRGQEPAKCPHWKNCL
TKLLPCFLEHNMKRDEDPHKAAKEMPFQGSGKSAWCPVEISKTVLWPESI
SVVRCVELFEAPVECEEEEEVEEEKGSFCASPESSRDDFQEGREGIVARL
TESLFLDLLGEENGGFCQQDMGESCLLPPSGSTSAHMPWDEFPSAGPKEA
PPWGKEQPLHLEPSPPASPTQSPDNLTCTETPLVIAGNPAYRSFSNSLSQ
SPCPRELGPDPLLARHLEEVEPEMPCVPQLSEPTTVPQPEPETWEQILRR
NVLQHGAAAAPVSAPTSGYQEFVHAVEQGGTQASAVVGLGPPGEAGYKAF
SSLLASSAVSPEKCGFGASSGEEGYKPFQDLIPGCPGDPAPVPVPLFTFG
LDREPPRSPQSSHLPSSSPEHLGLEPGEKVEDMPKPPLPQEQATDPLVDS
LGSGIVYSALTCHLCGHLKQCHGQEDGGQTPVMASPCCGCCCGDRSSPPT
TPLRAPDPSPGGVPLEASLCPASLAPSGISEKSKSSSSFHPAPGNAQSSS
QTPKIVNFVSVGPTYMRVS
IL3RB (AA 461 to 897 of NP_000386.1)
SEQ ID NO: 5
RFCGIYGYRLRRKWEEKIPNPSKSHLFQNGSAELWPPGSMSAFTSGSPPH
QGPWGSRFPELEGVFPVGFGDSEVSPLTIEDPKHVCDPPSGPDTTPAASD
LPTEQPPSPQPGPPAASHTPEKQASSFDFNGPYLGPPHSRSLPDILGQPE
PPQEGGSQKSPPPGSLEYLCLPAGGQVQLVPLAQAMGPGQAVEVERRPSQ
GAAGSPSLESGGGPAPPALGPRVGGQDQKDSPVAIPMSSGDTEDPGVASG
YVSSADLVFTPNSGASSVSLVPSLGLPSDQTPSLCPGLASGPPGAPGPVK
SGFEGYVELPPIEGRSPRSPRNNPVPPEAKSPVLNPGERPADVSPTSPQP
EGLLVLQQVGDYCFLPGLGPGPLSLRSKPSSPGPGPEIKNLDQAFQVKKP
PGQAVPQVPVIQLFKALKQQDYLSLPPWEVNKPGEVC
IL17RB (AA 314 to 502 of NP_061195.2)
SEQ ID NO: 6
RHERIKKTSFSTTTLLPPIKVLVVYPSEICFHHTICYFTEFLQNHCRSEV
ILEKWQKKKIAEMGPVQWLATQKKAADKVVFLLSNDVNSVCDGTCGKSEG
SPSENSQDLFPLAFNLFCSDLRSQIHLHKYVVVYFREIDTKDDYNALSVC
PKYHLMKDATAFCAELLHVKQQVSAGKRSQACHDGCCSL

The CAR endodomain may comprise a STAT5 association motif that comprises an amino acid sequence shown as SEQ ID NO: 1-7, or a variant which is at least 80, 85, 90, 95, 96, 97, 98 or 99% identical to SEQ ID NO: 1-7. For example, the variant may be capable of binding STAT5 to at least 10%, at least 20%, at least 30%, at least 40%, at least 50%, at least 60%, at least 70%, at least 80%, or at least 90% of the level of an amino acid sequence shown as one of SEQ ID NO: 1-7. The variant or derivative may be capable of binding STAT5 to a similar or the same level as one of SEQ ID NO: 1-7 or may be capable of binding STAT5 to a greater level than an amino acid sequence shown as one of SEQ ID NO: 1-7 (e.g. increased by at least 10%, at least 20%, at least 30%, at least 40% or at least 50%).

For example, the STAT5 association motif may be from IL2Rβ, IL7Rα, IL-3Rβ (CSF2RB), IL-9R, IL-17Rβ, erythropoietin receptor, thrombopoietin receptor, growth hormone receptor and prolactin receptor.

The STAT5 association motif may comprise the amino acid motif YXXF/L (SEQ ID NO: 8); wherein X is any amino acid.

Suitably, the STAT5 association motif may comprise the amino acid motif YCTF (SEQ ID NO: 9), YFFF (SEQ ID NO: 10), YLSL (SEQ ID NO: 11), or YLSLQ (SEQ ID NO: 12).

Suitably, the STAT5 association motif may comprise the amino acid motif YLSLQ (SEQ ID NO: 12).

The CAR endodomain may comprise one or more STAT5 association motif comprising the amino acid motif YCTF (SEQ ID NO: 9), YFFF (SEQ ID NO: 10), YLSL (SEQ ID NO: 11), and/or YLSLQ (SEQ ID NO: 12).

The CAR endodomain may comprise a first STAT5 association motif comprising the amino acid motif YLSLQ (SEQ ID NO: 12) and a second STAT5 association motif comprising the amino acid motif YCTF (SEQ ID NO: 9) or YFFF (SEQ ID NO: 10).

The CAR endodomain may comprise the following STAT5 association motifs: YLSLQ (SEQ ID NO: 12), YCTF (SEQ ID NO: 9) and YFFF (SEQ ID NO: 10).

“JAK1- and/or a JAK2-binding motif” as used herein refers to BOX motif which allows for tyrosine kinase JAK1 and/or JAK2 association. Suitable JAK1- and JAK2-binding motifs are described, for example, by Ferrao & Lupardus (Frontiers in Endocrinology; 2017; 8(71); which is incorporated herein by reference).

The JAK1 and/or JAK2-binding motif may occur endogenously in a cytoplasmic domain of a transmembrane protein.

For example, the JAK1 and/or JAK2-binding motif may be from Interferon lambda receptor 1 (IFNLR1), Interferon alpha receptor 1 (IFNAR), Interferon gamma receptor 1 (IFNGR1), IL10RA, IL20RA, IL22RA, Interferon gamma receptor 2 (IFNGR2) or IL10RB.

The JAK1-binding motif may comprise an amino acid motif shown as SEQ ID NO: 13-19 or a variant therefore which is capable of binding JAK1.

(SEQ ID NO: 13)
KVLKCNTPDPSKFFSQLSSEHGGDVQKWLSSPFPSSSFSPGGLAPEISPL
EVLERDK
(SEQ ID NO: 14)
NPWFQRAKMPRALDFSGHTHPVATFQPSRPESVNDLFLCPQKELT
(SEQ ID NO: 15)
GYICLRNSLPKVLNFHNFLAWPFPNLPPLEAMDMVEVIYINR
(SEQ ID NO: 16)
PLKEKSIILPKSLISVVRSATLETKPESKYVSLITSYQPFSL
(SEQ ID NO: 17)
RRRKKLPSVLLFKKPSPFIFISQRPSPETQDTIHPLDEEAFLK
(SEQ ID NO: 18)
YIHVGKEKHPANLILIYGNEFDKRFFVPAEKIVINFITLNISDDS
(SEQ ID NO: 19)
RYVTKPPAPPNSLNVQRVLTFQPLRFIQEHVLIPVFDLSGP

The variant of SEQ ID NO: 13-19 may comprise one, two or three amino acid differences compared to any of SEQ ID NO: 13-19 and retain the ability to bind JAK1.

The variant may be at least 80, 85, 90, 95, 96, 97, 98 or 99% identical to any one of SEQ ID NO: 13-19 and retain the ability to bind JAK1.

In a preferred embodiment, the JAK1-binding domain comprises SEQ ID NO: 13 or a variant thereof which is capable of binding JAK1.

For example, the variant may be capable of binding JAK1 to at least 10%, at least 20%, at least 30%, at least 40%, at least 50%, at least 60%, at least 70%, at least 80%, or at least 90% of the level of a corresponding, reference sequence. The variant or derivative may be capable of binding JAK1 to a similar or the same level as a corresponding, reference sequence or may be capable of binding JAK1 to a greater level than a corresponding, reference sequence (e.g. increased by at least 10%, at least 20%, at least 30%, at least 40% or at least 50%).

The JAK2-binding motif may comprise an amino acid motif shown as SEQ ID NO: 20-22 or a variant therefore which is capable of binding JAK2.

(SEQ ID NO: 20)
NYVFFPSLKPSSSIDEYFSEQPLKNLLLSTSEEQIEKCFIIEN
(SEQ ID NO: 21)
YWFHTPPSIPLQIEEYLKDPTQPILEALDKDSSPKDDVWDSVSIISFPE
(SEQ ID NO: 22)
YAFSPRNSLPQHLKEFLGHPHHNTLLFFSFPLSDENDVFDKLSVIAEDSE
S

The variant of SEQ ID NO: 21-22 may comprise one, two or three amino acid differences compared to any of SEQ ID NO: 20-22 and retain the ability to bind JAK2.

For example, the variant may be capable of binding JAK2 to at least 10%, at least 20%, at least 30%, at least 40%, at least 50%, at least 60%, at least 70%, at least 80%, or at least 90% of the level of a corresponding, reference sequence. The variant or derivative may be capable of binding JAK2 to a similar or the same level as a corresponding, reference sequence or may be capable of binding JAK2 to a greater level than a corresponding, reference sequence (e.g. increased by at least 10%, at least 20%, at least 30%, at least 40% or at least 50%).

Any method known in the art for determining protein:protein interactions may be used to determine whether a JAK1- or JAK2-binding motif is capable of binding to a JAK1 or JAK2. For example, co-immunoprecipitation followed by western blot

Suitably, the endodomain of the CAR described herein may not comprise a “Signal Transducer and Activator of Transcription 3” (STAT3) association motif.

STAT3 has been described as a detrimental signal for the stability and function of Tregs. For example, STAT3 signalling promotes the expression of pro-inflammatory genes such IL17, IL21, and IL22. As such, the use of a CAR which does not comprise a STAT3 association motif provides particular advantages in the context of an engineered Treg of the present invention.

A STAT3 association motif may comprise the amino acid sequence YXXQ (SEQ ID NO: 52), wherein “X” is any amino acid, and be capable of binding STAT3. Any method known in the art for determining protein:protein interactions may be used to determine whether a STAT3 association motif is capable of binding to STAT3. For example, co-immunoprecipitation followed by western blot.

Suitably, the CAR endodomain does not comprise the amino acid sequence YXXQ (SEQ ID NO: 52), wherein “X” is any amino acid.

“STAT3 association motif” may refer to an amino acid motif which comprises a tyrosine and is capable of binding a STAT3 polypeptide. For example, a “STAT3 association motif” as used herein may refer to an amino acid motif which comprises a tyrosine and is capable of functionally binding (i.e. leading to activation of) a STAT3 polypeptide, when present in a Treg.

Suitably, the CAR endodomain does not comprise an amino acid motif which comprises a tyrosine and is capable of binding a STAT3 polypeptide. For example, suitably the CAR endodomain does not comprise an amino acid motif which comprises a tyrosine and is capable of functionally binding (i.e. leading to activation of) a STAT3 polypeptide, when present in a Treg.

Suitably, the endodomain of the present CAR my not be capable of inducing productive STAT3 and/or STAT1 signalling when expressed in a Treg. In other words, when expressed in a Treg, the present CAR may not be capable of functionally binding and/or inducing phosphorylation and activation of STAT3 and/or STAT1. Suitably, the CAR may not be capable of inducing STAT3 and/or STAT1 dependent transcriptional activation when expressed in a Treg.

Suitably, the IL2Rβ endodomain portion of the CAR endodomain does not comprise a STAT3 association motif as defined herein.

Suitably, the CAR endodomain may comprise an IL2Rβ endodomain shown as SEQ ID NO: 1; or a variant which has at least 80% sequence identity to SEQ ID NO: 1.

SEQ ID NO: 1
NCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGED
DAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSR
PPGQGEFRALNARLPLNTDAYLSLQELQGQDPTHLV

The variant may be at least 80, 85, 90, 95, 96, 97, 98 or 99% identical to SEQ ID NO: 1.

Suitably, the CAR endodomain may comprise a truncated IL2Rβ endodomain shown as any one of SEQ ID NO: 23 or 24; or a variant of any one of SEQ ID NO: 23 or 24 which has at least 80% sequence identity thereto.

(IL2RB truncated - Y510)
SEQ ID NO: 23
NCRNTGPWLKKVLKCNTPDPSKFFSQLSSEHGGDVQKWLSSPFPSSSFSP
GGLAPEISPLEVLERDKVTQLLPLNTDAYLSLQELQGQDPTHLV
(IL2RB truncated - Y510 & Y392)
SEQ ID NO: 24
NCRNTGPWLKKVLKCNTPDPSKFFSQLSSEHGGDVQKWLSSPFPSSSFSP
GGLAPEISPLEVLERDKVTQLLDAYCTFPSRDDLLLFSPSLLGGPSPPST
APGGSGAGEERMPPSLQERVPRDWDPQPLGPPTPGVPDLVDFQPPPELVL
REAGEEVPDAGPREGVSFPWSRPPGQGEFRALNARLPLNTDAYLSLQELQ
GQDPTHLV

The variant may be at least 80, 85, 90, 95, 96, 97, 98 or 99% identical to SEQ ID NO: 23 or 24.

STAT5 activity is increased by the transphosphorylation of both a Jak1/2 and Jak3, as this stabilizes their activity. Suitably, the CAR endodomain as described herein may further comprise a JAK3-binding motif. “JAK3-binding motif” as used herein refers to BOX motif which allows for tyrosine kinase JAK3. Suitable JAK3-binding motifs are described, for example, by Ferrao & Lupardus (Frontiers in Endocrinology; 2017; 8(71); which is incorporated herein by reference).

Any method known in the art for determining protein:protein interactions may be used to determine whether a motif is capable of binding to JAK3. For example, co-immunoprecipitation followed by western blot.

The JAK3-binding motif may occur endogenously in a cytoplasmic domain of a transmembrane protein.

For example, the JAK3-binding motif may be from an IL-2Rγ polypeptide.

The JAK3-binding motif may comprise an amino acid motif shown as SEQ ID NO: 25 or SEQ ID NO: 26 or a variant therefore which is capable of binding JAK3.

SEQ ID NO: 25
ERTMPRIPTLKNLEDLVTEYHGNFSAWSGVSKGLAESLQPDYSERLCLVS
EI
SEQ ID NO: 26
ERTMPRIPTLKNLEDLVTEYHGNFSAWSGVSKGLAESLQPDYSERLCLVS
EIPPKGGALGEGPGASPCNQHSPYWAPPCYTLKPET

The variant may be at least 80, 85, 90, 95, 96, 97, 98 or 99% identical to SEQ ID NO: 25 or SEQ ID NO: 26.

In a preferred embodiment, the CAR endodomain comprises one or more JAK1-binding domains and at least one JAK3-binding domain.

The endodomain of a CAR as described herein also comprises motifs necessary to transduce the effector function signal and direct the Treg to perform its specialized function upon antigen binding. Examples of intracellular signaling domains include, but are not limited to, ζ chain endodomain of the T-cell receptor or any of its homologs (e.g., η chain, FcεR1γ and β chains, MB1 (Igα) chain, B29 (Igβ) chain, etc.), CD3 polypeptide domains (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may comprise human CD3 zeta chain endodomain, FcyRIII, FcsRI, cytoplasmic tails of Fc receptors, immunoreceptor tyrosine-based activation motif (ITAM) bearing cytoplasmic receptors or combinations thereof.

Preferably, the intracellular signaling domain comprises the intracellular signaling domain of a human CD3 zeta chain.

In one embodiment the intracellular signaling domain of human CD3 zeta chain comprises the following sequence:

UNIPROT: P20963, CD3Z_HUMAN, position 31-143
(SEQ ID NO: 27)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR

In one embodiment, the intracellular signaling domain comprises at least 85, 90, 95, 97, 98 or 99% identity to SEQ ID NO: 27.

The intracellular signaling domain of the CAR may comprise the following CD28 signaling domain:

(SEQ ID NO: 28)
RSKRSRLLHSDYMNMTPRRPGPTRKHYQPYAPPRDFAAYRS

In one embodiment, the intracellular signaling domain a signaling motif which has at least 85, 90, 95, 97, 98 or 99% identity to SEQ ID NO: 28.

The intracellular signaling domain of the CAR may comprise the following CD27 signaling domain

(SEQ ID NO: 29)
QRRKYRSNKGESPVEPAEPCHYSCPREEEGSTIPIQEDYRKPEPACSP.

In one embodiment, the intracellular signaling domain a signaling motif which has at least 85, 90, 95, 97, 98 or 99% identity to SEQ ID NO: 29.

Additional intracellular signaling domains will be apparent to those of skill in the art and may be used in connection with alternate embodiments of the invention.

The present CAR may comprise a compound endodomain comprising a fusion of the intracellular part of a T-cell co-stimulatory molecule to that of e.g. CD3. Such a compound endodomain may be referred to as a second generation CAR which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. The CAR endodoman may also comprise one or more TNF receptor family signalling domain, such as the signalling domain of OX40, 4-1BB, ICOS or TNFRSF25.

Illustrative sequences for OX40, 4-1BB, ICOS and TNFRSF25 signalling domains are shown below as SEQ ID NO: 30-33. The CAR endodoman may also comprise one or more of SEQ ID NO: 30-33 or a variant of SEQ ID NO: 30-33.

OX40 signalling domain (SEQ ID NO: 30):
ALYLLRRDQRLPPDAHKPPGGGSFRTPIQEEQADAHSTLAKI
41BB signalling domain (SEQ ID NO: 31):
KRGRKKLLYIFKQPFMRPVQTTQEEDGCSCRFPEEEEGGCEL
ICOS signalling domain (SEQ ID NO: 32):
CWLTKKKYSSSVHDPNGEYMFMRAVNTAKKSRLTDVTL
TNFRSF25 signalling domain (SEQ ID NO: 33):
TYTYRHCWPHKPLVTADEAGMEALTPPPATHLSPLDSAHTLLAPPDSSEK
ICTVQLVGNSWTPGYPETQEALCPQVTWSWDQLPSRALGPAAAPTLSPES
PAGSPAMMLQPGPQLYDVMDAVPARRWKEFVRTLGLREAEIEAVEVEIGR
FRDQQYEMLKRWRQQQPAGLGAVYAALERMGLDGCVEDLRSRLQRGP

The CAR endodoman may comprise a variant of one or more of SEQ ID NO: 30-33 which has at least 85, 90, 95, 97, 98 or 99% identity to any one of SEQ ID NO: 30-33.

Suitably, the CAR endodomain may comprise SEQ ID NO: 45 or a variant which has at least 85, 90, 95, 97, 98 or 99% identity to SEQ ID NO: 45.

(illustrative endodomain sequence comprising CD28,
IL2RG-T52, IL2RB-Y510, CD3 zeta signalling
domains)
SEQ ID NO: 45
RSKRSRLLHSDYMNMTPRRPGPTRKHYQPYAPPRDFAAYRSERTMPRIPT
LKNLEDLVTEYHGNFSAWSGVSKGLAESLQPDYSERLCLVSEINCRNTGP
WLKKVLKCNTPDPSKFFSQLSSEHGGDVQKWLSSPFPSSSFSPGGLAPEI
SPLEVLERDKVTQLLPLNTDAYLSLQELQGQDPTHLVRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR

Suitably, the CAR endodomain may comprise SEQ ID NO: 53 or a variant which has at least 85, 90, 95, 97, 98 or 99% identity to SEQ ID NO: 53.

(illustrative endodomain sequence comprising CD28,
IL2RG-T52, IL7RA-2Y, CD3 zeta signalling domains
SEQ ID NO: 53
RSKRSRLLHSDYMNMTPRRPGPTRKHYQPYAPPRDFAAYRSERTMPRIPT
LKNLEDLVTEYHGNFSAWSGVSKGLAESLQPDYSERLCLVSEIKKRIKPI
VWPSLPDHKKTLEHLCKKPRKNLNVSFNPESFLDCQIHRVDDIQARDEVE
GFLQDTFPQQPILTSLGSNQEEAYVTMSSFYQNQRVKFSRSADAPAYQQG
QNQLYNELNLGRREEYDVLDKRRGRDPEMGGKPRRKNPQEGLYNELQKDK
MAEAYSEIGMKGERRRGKGHDGLYQGLSTATKDTYDALHMQALPPRGSGA
TNFSLLKQAGDVEENPG

Variants, Derivatives and Fragments

In addition to the specific proteins, peptides and nucleotides mentioned herein, the present invention also encompasses the use of derivatives and fragments thereof.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains the desired function (for example, where the derivative or variant is an antigen binding domain, the desired function may be the ability of the antigen binding domain to bind its target antigen, or where the derivative or variant is a signalling domain, the desired function may be the ability of that domain to signal (e.g. activate or inactivate a downstream molecule). For example, variant or derivative may have at least at least 10%, at least 20%, at least 30%, at least 40%, at least 50%, at least 60%, at least 70%, at least 80%, or at least 90% function compared to the corresponding, reference sequence. The variant or derivative may have a similar or the same level of function as compared to the corresponding, reference sequence or may have an increased level of function (e.g. increased by at least 10%, at least 20%, at least 30%, at least 40% or at least 50%).

Typically, amino acid substitutions may be made, for example from 1, 2 or 3 to 10 or 20 substitutions provided that the modified sequence retains the required activity or ability. Amino acid substitutions may include the use of non-naturally occurring analogues. For example, the variant or derivative may have at least 10%, at least 20%, at least 30%, at least 40%, at least 50%, at least 60%, at least 70%, at least 80%, or at least 90% activity or ability compared to the corresponding, reference sequence. The variant or derivative may have a similar or the same level of activity or ability as compared to the corresponding, reference sequence or may have an increased level of activity or ability (e.g. increased by at least 10%, at least 20%, at least 30%, at least 40% or at least 50%).

Proteins or peptides used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

The derivative may be a homolog. The term “homologue” as used herein means an entity having a certain homology with the wild type amino acid sequence and the wild type nucleotide sequence. The term “homology” can be equated with “identity”.

A homologous or variant sequence may include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence.

Once the software has produced an optimal alignment, it is possible to calculate percentage homology, preferably percentage sequence identity. The software typically does this as part of the sequence comparison and generates a numerical result.

“Fragments” typically refers to a selected region of the polypeptide or polynucleotide that is of interest functionally. “Fragment” thus refers to an amino acid or nucleic acid sequence that is a portion of a full-length polypeptide or polynucleotide.

Such derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Antigen-Specific Targeting Domain

The antigen-specific targeting domain provides the CAR with the ability to bind a predetermined antigen of interest. The antigen-specific targeting domain preferably targets an antigen of clinical interest.

The antigen-specific targeting domain may be any protein or peptide that possesses the ability to specifically recognize and bind to a biological molecule (e.g., a cell surface receptor or a component thereof). The antigen-specific targeting domain includes any naturally occurring, synthetic, semi-synthetic, or recombinantly produced binding partner for a biological molecule of interest. Illustrative antigen-specific targeting domains include antibodies or antibody fragments or derivatives, extracellular domains of receptors, ligands for cell surface molecules/receptors, or receptor binding domains thereof, and tumor binding proteins. Although as discussed below, the antigen-specific targeting domain may preferably be an antibody or derived from an antibody, other antigen-specific targeting domains are encompassed, e.g. antigen-specific targeting domains formed from an antigenic peptide/MHC or HLA combination which is capable of binding to the TCRs of Tcon cells active at a site of transplantation, inflammation or disease. Such antigen-binding domains have been reported for example in Mekala et al, Blood, 2005, vol 105, pages 2090-2092.

In a preferred embodiment, the antigen-specific targeting domain is, or is derived from, an antibody. An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VH), a light chain variable region (VL) a camelid antibody (VHH) and a single domain antibody (sAb).

In a preferred embodiment, the binding domain is a single chain antibody (scFv). The scFv may be murine, human or humanized scFv.

“Complementarity determining region” or “CDR” with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs. “Heavy chain variable region” or “VH” refers to the fragment of the heavy chain of an antibody that contains three CDRs interposed between flanking stretches known as framework regions, which are more highly conserved than the CDRs and form a scaffold to support the CDRs. “Light chain variable region” or “VL” refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain. “Single-chain Fv antibody” or “scFv” refers to an engineered antibody consisting of a light chain variable region and a heavy chain variable region connected to one another directly or via a peptide linker sequence.

Antibodies that specifically bind a predetermined antigen can be prepared using methods well known in the art. Such methods include phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined.

Antigens which may be targeted by the present CAR include, but are not limited to, antigens expressed on cells associated with transplanted organs, autoimmune diseases, allergic diseases and inflammatory diseases. It will be understood by a skilled person that due to the bystander effect of Treg cells, the antigen may be simply present and/or expressed at the site of transplantation, inflammation or disease.

Antigens associated with organ transplants and/or cells associated with transplanted organs include, but are not limited to, a HLA antigen present in the transplanted organ but not in the patient, or an antigen whose expression is up-regulated during transplant rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

By way of example, the CAR may comprise an antigen binding domain which is capable of binding HLA-A2 (HLA-A2 may also be referred to herein as HLA-A*02, HLA-A02, and HLA-A*2). HLA-A*02 is one particular class I major histocompatibility complex (MHC) allele group at the HLA-A locus.

The antigen recognition domain may bind, suitably specifically bind, one or more region or epitope within HLA-A2. An epitope, also known as antigenic determinant, is the part of an antigen that is recognised by an antigen recognition domain (e.g. an antibody). In other words, the epitope is the specific piece of the antigen to which an antibody binds. Suitably, the antigen recognition domain binds, suitably specifically binds, to one region or epitope within HLA-A2.

The antigen recognition domain may comprise at least one CDR (e.g. CDR3), which can be predicted from an antibody which binds to an antigen, preferably HLA-A2 (or a variant of such a predicted CDR (e.g. a variant with one, two or three amino acid substitutions)). It will be appreciated that molecules containing three or fewer CDR regions (e.g. a single CDR or even a part thereof) may be capable of retaining the antigen-binding activity of the antibody from which the CDR is derived. Molecules containing two CDR regions are described in the art as being capable of binding to a target antigen, e.g. in the form of a minibody (Vaughan and Sollazzo, 2001, Combinational Chemistry & High Throughput Screening, 4, 417-430). Molecules containing a single CDR have been described which can display strong binding activity to target (Nicaise et al, 2004, Protein Science, 13: 1882-91).

In this respect, the antigen recognition domain may comprise one or more variable heavy chain CDRs, e.g. one, two or three variable heavy chain CDRs. Alternatively, or additionally, the antigen recognition domain may comprise one or more variable light chain CDRs, e.g. one, two or three variable light chain CDRs. The antigen recognition domain may comprise three heavy chain CDRs and/or three light chain CDRs (and more particularly a heavy chain variable region comprising three CDRs and/or a light chain variable region comprising three CDRs) wherein at least one CDR, preferably all CDRs, may be from an antibody which binds to an antigen, preferably HLA-A2, or may be selected from one of the CDR sequences provided below.

The antigen recognition domain may comprise any combination of variable heavy and light chain CDRs, e.g. one variable heavy chain CDR together with one variable light chain CDR, two variable heavy chain CDRs together with one variable light chain CDR, two variable heavy chain CDRs together with two variable light chain CDRs, three variable heavy chain CDRs together with one or two variable light chain CDRs, one variable heavy chain CDR together with two or three variable light chain CDRs, or three variable heavy chain CDRs together with three variable light chain CDRs. Preferably, the antigen recognition domain comprises three variable heavy chain CDRs (CDR1, CDR2 and CDR3) or three variable light chain CDRs (CDR1, CDR2 and CDR3).

The one or more CDRs present within the antigen recognition domain may not all be from the same antibody, as long as the domain has the binding activity described above. Thus, one CDR may be predicted from the heavy or light chains of an antibody which binds to an antigen, e.g. HLA-A2 whilst another CDR present may be predicted from a different antibody which binds to the same antigen (e.g. HLA-A2). In this instance, it may be preferred that CDR3 be predicted from an antibody that binds to an antigen, e.g. HLA-A2. Particularly however, if more than one CDR is present in the antigen recognition domain, it is preferred that the CDRs are predicted from antibodies which bind to the same antigen, e.g. HLA-A2. A combination of CDRs may be used from different antibodies, particularly from antibodies that bind to the same desired region or epitope.

In a particularly preferred embodiment, the antigen recognition domain comprises three CDRs predicted from the variable heavy chain sequence of an antibody which binds to aa antigen, e.g. HLA-A2 and/or three CDRs predicted from the variable light chain sequence of an antibody which binds to an antigen, e.g. HLA-A2 (preferably the same antibody).

In some embodiments, the antigen recognition domain is, or is derived from an antibody (e.g. is a Fab, scFv, or sdAb) wherein the antibody comprises one or more CDR regions, selected from SEQ ID NOs: 90-146, or derivatives thereof. In other words, in some embodiments the antigen recognition domain comprises one or more CDR regions, selected from SEQ ID NOs: 90-146, or derivatives thereof. Suitably, the antigen recognition domain comprises three CDR regions selected from SEQ ID NOs: 90-146, or derivatives thereof.

| Name | CDR1 | CDR2 | CDR3 |
|---|---|---|---|
| GL VH CDRs | DYGMH | FIRNDGSDKYYADSVKG | NGESGPLDYWYFDL |
| (SEQ ID NOs: 90-92) | (SEQ ID NO: 90) | (SEQ ID NO: 91) | (SEQ ID NO: 92) |
| 3PB2 VH CDRs | DYGMH | FIRNDGSDKYYADSVKG | NGESGPLDYWYLDL |
| (SEQ ID NOs: 93-95) | (SEQ ID NO: 93) | (SEQ ID NO: 94) | (SEQ ID NO: 95) |
| 3PC4 VH CDRs | DYGMH | FIRNDGSDKYYADSVRG | NGESGPLDYWYFDL |
| (SEQ ID NOs: 96-98) | (SEQ ID NO: 96) | (SEQ ID NO: 97) | (SEQ ID NO: 98) |
| 3PF12 VH CDRs | DYGMH | FIRNDGSDKYYADSVKG | NGESGPLDYWYFDL |
| (SEQ ID NOs: 99-101) | (SEQ ID NO: 99) | (SEQ ID NO: 100) | (SEQ ID NO: 101) |
| GL VL CDRs | QASQDISNYLN | DASNLET | QQYDNLPPT |
| (SEQ ID NOs: 102-104) | (SEQ ID NO: 102) | (SEQ ID NO: 103) | (SEQ ID NO: 104) |
| 3PB2 VL CDRs | QSSLDISHYLN | DASNLET | QQYDNLPLT |
| (SEQ ID NOs: 105-107) | (SEQ ID NO: 105) | (SEQ ID NO: 106) | (SEQ ID NO: 107) |
| 3PC4 VL CDRs | RASHGINNYLA | AASTLQS | QQYDSYPPT |
| (SEQ ID NOs: 108-110) | (SEQ ID NO: 108) | (SEQ ID NO: 109) | (SEQ ID NO: 110) |
| 3PF12 VL CDRs | QASQDISNYLN | DASNLET | QQYSSFPLT |
| (SEQ ID NOs: 111-113) | (SEQ ID NO: 111) | (SEQ ID NO: 112) | (SEQ ID NO: 113) |
| C12 VL CDRs | QASQDISNYLN | DETHLDS | QQYDSLPPT |
| (SEQ ID NOs: 114-116) | (SEQ ID NO: 114) | (SEQ ID NO: 115) | (SEQ ID NO: 116) |
| E7 VL CDRs | QASQDISNYLN | DASNLET | QQYDNLPIT |
| (SEQ ID NOs: 117-119) | (SEQ ID NO: 117) | (SEQ ID NO: 118) | (SEQ ID NO: 119) |
| H10 VL CDRs | QASQDISNYLN | DASNLET | QQYDNLPST |
| (SEQ ID NOs: 120-122) | (SEQ ID NO: 120) | (SEQ ID NO: 121) | (SEQ ID NO: 122) |
| B8 VL CDRs | QASQDISNYLN | DASNLET | QQYNTYPLT |
| (SEQ ID NOs: 123-125) | (SEQ ID NO: 123) | (SEQ ID NO: 124) | (SEQ ID NO: 125) |
| D2 VL CDRs | QASQDISNYLN | DASNLET | QQYHTYPLT |
| (SEQ ID NOs: 126-128) | (SEQ ID NO: 126) | (SEQ ID NO: 127) | (SEQ ID NO: 128) |
| B10 VL CDRs | QASQDISNYLN | DASNLET | QQYDNLPLT |
| (SEQ ID NOs: 129-131) | (SEQ ID NO: 129) | (SEQ ID NO: 130) | (SEQ ID NO: 131) |
| 2A9 VL CDRs | RTSQGISSALA | DASSLES | QQFNNYPLT |
| (SEQ ID NOs: 132-134) | (SEQ ID NO: 132) | (SEQ ID NO: 133) | (SEQ ID NO: 134) |
| 3B12 VL CDRs | QASQDISNYLA | AASNLQS | LQDSSYPPT |
| (SEQ ID NOs: 135-137) | (SEQ ID NO: 135) | (SEQ ID NO: 136) | (SEQ ID NO: 137) |
| 2D4 VL CDRs | RASQSISSWLA | KASNLQS | QQYSNYPLT |
| (SEQ ID NOs: 138-140) | (SEQ ID NO: 138) | (SEQ ID NO: 139) | (SEQ ID NO: 140) |
| 3D4 VL CDRs | RASHGISNYFA | ATSTLQS | QQYSSYPLT |
| (SEQ ID NOs: 141-143) | (SEQ ID NO: 141) | (SEQ ID NO: 142) | (SEQ ID NO: 143) |
| B3 VL CDRs | RASRGSNYLA | ATSTLQS | QQYDSYPPT |
| (SEQ ID NOs: 144-146) | (SEQ ID NO: 144) | (SEQ ID NO: 145) | (SEQ ID NO: 146) |

Preferably, the antigen binding domain comprises CDRs (CDR1, CDR2, and CDR3), or derivatives thereof, selected from the same variable chain. For example, the antigen binding domain may comprise SEQ ID NOs: 90-92, SEQ ID NOs: 93-95, SEQ ID NOs: 96-98, SEQ ID NOs: 99-101, SEQ ID NOs: 102-104, SEQ ID NOs: 105-107, SEQ ID NOs: 108-110, SEQ ID NOs: 111-113, SEQ ID NOs: 114-116, SEQ ID NOs: 117-119, SEQ ID NOs: 120-122, SEQ ID NOs: 123-125, SEQ ID NOs: 126-128, SEQ ID NOs: 129-131, SEQ ID NOs: 132-134, SEQ ID NOs: 135-137, SEQ ID NOs: 138-140, SEQ ID NOs: 141-143, and/or SEQ ID NOs: 144-146, or derivatives thereof.

In preferred embodiments, the antigen recognition domain comprises a combination variable heavy and variable light CDRs as follows:
(i) SEQ ID NOs: 90-92 and SEQ ID NOs: 102-104, or derivatives thereof;
(ii) SEQ ID NOs: 93-95 and SEQ ID NOs: 105-107, or derivatives thereof;
(iii) SEQ ID NOs: 96-98 and SEQ ID NOs: 108-110, or derivatives thereof;
(iv) SEQ ID NOs: 99-101 and SEQ ID NOs: 111-113, or derivatives thereof;
(v) SEQ ID NOs: 99-101 and SEQ ID NOs: 114-116, or derivatives thereof;
(vi) SEQ ID NOs: 99-101 and SEQ ID NOs: 117-119, or derivatives thereof;
(vii) SEQ ID NOs: 99-101 and SEQ ID NOs: 120-122, or derivatives thereof;
(viii) SEQ ID NOs: 99-101 and SEQ ID NOs: 123-125, or derivatives thereof;
(ix) SEQ ID NOs: 99-101 and SEQ ID NOs: 126-128, or derivatives thereof;
(x) SEQ ID NOs: 99-101 and SEQ ID NOs: 129-131, or derivatives thereof;
(xi) SEQ ID NOs: 99-101 and SEQ ID NOs: 132-134, or derivatives thereof;
(xii) SEQ ID NOs: 99-101 and SEQ ID NOs: 135-137, or derivatives thereof;
(xiii) SEQ ID NOs: 99-101 and SEQ ID NOs: 138-140, or derivatives thereof;
(xiv) SEQ ID NOs: 99-101 and SEQ ID NOs: 141-143, or derivatives thereof;
(xv) SEQ ID NOs: 99-101 and SEQ ID NOs: 144-146, or derivatives thereof;

Preferably, the antigen recognition domain comprises SEQ ID NOs: 93-95 and SEQ ID NOs: 105-107, or derivatives thereof.

The antigen binding domain may comprise a variable heavy domain selected from SEQ ID NO: 54, 55, 56 or 57 or a variant which is at least 80% identical to SEQ ID NO: 54, 55, 56 or 57. The variant which may be at least 85, 90, 95, 97, 98 or 99% identical to SEQ ID NO: 54, 55, 56 or 57.

SEQ ID NO: 54
VQLVQSGGGVVQPGGSLRVSCAASGVTLSDYGMHWVRQAPGKGLEWMAFI
RNDGSDKYYADSVKGRFTISRDNSKKTVSLQMSSLRAEDTAVYYCAKNGE
SGPLDYWYFDLWGRGTLVTV
SEQ ID NO: 55
QVQLVQSGGGVVQPGGSLRVSCAASGVTLSDYGMHWVRQAPGKGLEWVAF
IRNDGSDKYYADSVKGRFTISRDNSEKTVSLQMSSLRAEDTAVYYCAKNG
ESGPLDYWYLDLWGRGT
SEQ ID NO: 56
QVQLVQSGGGVVQPGGSMRVSCAASGVTLSDYGMHWVRQAPGKGLEWVAF
IRNDGSDKYYADSVRGRFTISRDNSKKTVFLQMNSLRAEDTAVYYCAKNG
ESGPLDYWYFDLWGRGT
SEQ ID NO: 57
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGT

The antigen binding domain may comprise a variable light domain selected from SEQ ID NO: 58 to 72 or a variant which is at least 80% identical to SEQ ID NO: 58 to 72. The variant which may be at least 85, 90, 95, 97, 98 or 99% identical to SEQ ID NO: 58 to 72.

SEQ ID NO: 58
DVVMTQSPSSLSASVGDRVTITCQASQDISNYLNWYQQKPGKAPKLLIYD
ASNLETGVPSRFSGSGSGTDFTFTISSLQPEDIATYYCQQYDNLPPTFGG
GTKLTVLG
SEQ ID NO: 59
DVVMTQSPSSLSASVGDRVTITCQSSLDISHYLNWYQQKPGKAPKLLIYD
ASNLETGVPSRFSGSGSGTHFTFTISSLQPEDFATYYCQQYDNLPLTFGG
GTKLEIK
SEQ ID NO: 60
DIVLMQSPSFLSASVGDRVTITCRASHGINNYLAWYQQKPGKAPKLLIYA
ASTLQSGVPSRFSGSGSGTEFTLTISSLQPEDFATYYCQQYDSYPPTFGR
TKVEIKR
SEQ ID NO: 61
DVVMTQSPSSLSASVGDRVTITCQASQDISNYLNWYQQKPGKAPKLLIYD
ASNLETGVPSRFSGSGSGTDFTFTISSLQPEDFATYYCQQYSSFPLTFGG
GTKVDIK
SEQ ID NO: 62
DVVMTQSPSSLSASVGDRVTITCQASQDISNYLNWYQQEPGKAPKLLIYD
ETHLDSGVPSRFTGSRSGTDFTLTISSLQPEDFATYYCQQYDSLPPTFGG
GTKVDIK
SEQ ID NO: 63
DVVMTQSPSSLSASVGDRVTITCQASQDISNYLNWYQQKPGKAPKLLIYD
ASNLETGVPSRFSGSGSGTDFTFTISSLQPEDIATYYCQQYDNLPITFGG
GTKVDIK
SEQ ID NO: 64
DVVMTQSPSSLSASVGDRVTITCQASQDISNYLNWYQQKPGKAPKLLIYD
ASNLETGVPSRFSGSGSGTDFTFTISSLQPEDIATYYCQQYDNLPSTFGG
GTKVDIK
SEQ ID NO: 65
DVVMTQSPSSLSASVGDRVTITCQASQDISNYLNWYQQKPGKAPKLLIYD
ASNLETGVPSRFSGSGSGTDFTFTISSLQPEDFGTYYCQQYNTYPLTFGG
GTKVDIK
SEQ ID NO: 66
DVVMTQSPSSLTASVGDRVTITCQASQDISNYLNWYQQKPGKAPKLLIYD
ASNLETGVPSRFSGSGSGTDFTLSIDSLQPEDFATYYCQQYHTYPLTFGG
GTKVDIK
SEQ ID NO: 67
DVVMTQSPSSLSASVGDRVTITCQASQDISNYLNWYQQKPGKAPKLLIYD
ASNLETGVPSRFSGSGSGTDFTFTISSLQPEDIATYYCQQYDNLPLTFGG
GTKVDIK
SEQ ID NO: 68
DVVMTQSPSSLSASVGDRVTITCRTSQGISSALAWYQQKPGKAPKLLIYD
ASSLESGVPSRFSGSGSGTDFTLTISSLQPEDFATYYCQQFNNYPLTFGG
GTKVDIK
SEQ ID NO: 69
DVVMTQSPSSLSASVGDRVTITCQASQDISNYLAWYQQKPGRAPTLLIFA
ASNLQSGVPSRFSGSGSGTEFTLTISGLQPEDFATYYCLQDSSYPPTFGG
GTKVDIK
SEQ ID NO: 70
DVVMTQSPSTLSASVGDRVTITCRASQSISSWLAWYQQKPGRAPTLLIYK
ASNLQSGVPSRFSGSGSGTEFTLTISSLQPDDFASYYCQQYSNYPLTFGG
GTKVDIK
SEQ ID NO: 71
DVVMTQSPSFLSASVGDRVTITCRASHGISNYFAWYQQKPGKAPKLLIYA
TSTLQSGVPSRFSGSGSGTEFTLTISGLQPEDFATYYCQQYSSYPLTFGG
GTKVDIK
SEQ ID NO: 72
DVVMTQSPSTLSAYVGDRITITCRASRGSNYLAWYQQKPGKAPKLLIYAT
STLQSGVPLRFSGSGSGTEFTLTISGLQPEDFATYYCQQYDSYPPTFGGG
TKVDIK

The antigen binding domain may comprise SEQ ID NO: 34, or 73-86 or a variant which is at least 80% identical to SEQ ID NO: 34, or 73-86 and is capable of binding to HLA-A2. The variant which may be at least 85, 90, 95, 97, 98 or 99% identical to SEQ ID NO: 34, or 73-86.

SEQ ID NO: 73
QVQLVQSGGGVVQPGGSLRVSCAASGVTLSDYGMHWVRQAPGKGLEWVAF
IRNDGSDKYYADSVKGRFTISRDNSEKTVSLQMSSLRAEDTAVYYCAKNG
ESGPLDYWYLDLWGSSGGGGSGGGGSGGGGSDVVMTQSPSSLSASVGDRV
TITCQSSLDISHYLNWYQQKPGKAPKLLIYDASNLETGVPSRFSGSGSGT
HFTFTISSLQPEDFATYYCQQYDNLPLTFGGGTKLEIK
SEQ ID NO: 34
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTLVTVSSGGGGSGGGGSGGGGSDVVMTQSPSSLS
ASVGDRVTITCQASQDISNYLNWYQQKPGKAPKLLIYDASNLETGVPSRF
SGSGSGTDFTFTISSLQPEDIATYYCQQYDNLPPTFGGGTKLTVLG
SEQ ID NO: 74
QVQLVQSGGGVVQPGGSMRVSCAASGVTLSDYGMHWVRQAPGKGLEWVAF
IRNDGSDKYYADSVRGRFTISRDNSKKTVFLQMNSLRAEDTAVYYCAKNG
ESGPLDYWYFDLWGRGTSSGGGGSGGGGSGGGGSDIVLMQSPSFLSASVG
DRVTITCRASHGINNYLAWYQQKPGKAPKLLIYAASTLQSGVPSRFSGSG
SGTEFTLTISSLQPEDFATYYCQQYDSYPPTFGRTKVEIKR
SEQ ID NO: 75
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQKPGKAPKLLIYDASNLETGVPSRFSGSG
SGTDFTFTISSLQPEDFATYYCQQYSSFPLTFGGGTKVDIK
SEQ ID NO: 76
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQEPGKAPKLLIYDETHLDSGVPSRFTGSR
SGTDFTLTISSLQPEDFATYYCQQYDSLPPTFGGGTKVDIK
SEQ ID NO: 77
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQKPGKAPKLLIYDASNLETGVPSRFSGSG
SGTDFTFTISSLQPEDIATYYCQQYDNLPITFGGGTKVDIK
SEQ ID NO: 78
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQKPGKAPKLLIYDASNLETGVPSRFSGSG
SGTDFTFTISSLQPEDIATYYCQQYDNLPSTFGGGTKVDIK
SEQ ID NO: 79
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQKPGKAPKLLIYDASNLETGVPSRFSGSG
SGTDFTFTISSLQPEDFGTYYCQQYNTYPLTFGGGTKVDIK
SEQ ID NO: 80
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLTASVG
DRVTITCQASQDISNYLNWYQQKPGKAPKLLIYDASNLETGVPSRFSGSG
SGTDFTLSIDSLQPEDFATYYCQQYHTYPLTFGGGTKVDIK
SEQ ID NO: 81
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLNWYQQKPGKAPKLLIYDASNLETGVPSRFSGSG
SGTDFTFTISSLQPEDIATYYCQQYDNLPLTFGGGTKVDIK
SEQ ID NO: 82
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCRTSQGISSALAWYQQKPGKAPKLLIYDASSLESGVPSRFSGSG
SGTDFTLTISSLQPEDFATYYCQQFNNYPLTFGGGTKVDIK
SEQ ID NO: 83
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSSLSASVG
DRVTITCQASQDISNYLAWYQQKPGRAPTLLIFAASNLQSGVPSRFSGSG
SGTEFTLTISGLQPEDFATYYCLQDSSYPPTFGGGTKVDIK
SEQ ID NO: 84
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSTLSASVG
DRVTITCRASQSISSWLAWYQQKPGRAPTLLIYKASNLQSGVPSRFSGSG
SGTEFTLTISSLQPDDFASYYCQQYSNYPLTFGGGTKVDIK
SEQ ID NO: 85
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSFLSASVG
DRVTITCRASHGISNYFAWYQQKPGKAPKLLIYATSTLQSGVPSRFSGSG
SGTEFTLTISGLQPEDFATYYCQQYSSYPLTFGGGTKVDIK
SEQ ID NO: 86
QVQLVQSGGGVVQPGGSLRVSCAASGVTLSDYGMHWVRQAPGKGLEWMAF
IRNDGSDKYYADSVKGRFTISRDNSKKTVSLQMSSLRAEDTAVYYCAKNG
ESGPLDYWYFDLWGRGTSSGGGGSGGGGSGGGGSDVVMTQSPSTLSAYVG
DRITITCRASRGSNYLAWYQQKPGKAPKLLIYATSTLQSGVPLRFSGSGS
GTEFTLTISGLQPEDFATYYCQQYDSYPPTFGGGTKVDIK

The antigen binding domain may comprise SEQ ID NO: 73, or a variant which is at least 80% identical to SEQ ID NO: 73. Suitably, the variant may be at least 85, 90, 95, 97, 98 or 99% identical to SEQ ID NO: 73.

The antigen binding domain may comprise SEQ ID NO: 34, or a variant which is at least 80% identical to SEQ ID NO: 34. Suitably, the variant may be at least 85, 90, 95, 97, 98 or 99% identical to SEQ ID NO: 34.

The antigen binding domain may comprise SEQ ID NO: 74, or a variant which is at least 80% identical to SEQ ID NO: 74. Suitably, the variant may be at least 85, 90, 95, 97, 98 or 99% identical to SEQ ID NO: 74.

The antigen binding domain may comprise SEQ ID NO: 75, or a variant which is at least 80% identical to SEQ ID NO: 75. Suitably, the variant may be at least 85, 90, 95, 97, 98 or 99% identical to SEQ ID NO: 75.

The antigen binding domain may comprise SEQ ID NO: 76, or a variant which is at least 80% identical to SEQ ID NO: 76. Suitably, the variant may be at least 85, 90, 95, 97, 98 or 99% identical to SEQ ID NO: 76.

The antigen binding domain may comprise SEQ ID NO: 77, or a variant which is at least 80% identical to SEQ ID NO: 77. Suitably, the variant may be at least 85, 90, 95, 97, 98 or 99% identical to SEQ ID NO: 77.

The antigen binding domain may comprise SEQ ID NO: 78, or a variant which is at least 80% identical to SEQ ID NO: 78. Suitably, the variant may be at least 85, 90, 95, 97, 98 or 99% identical to SEQ ID NO: 78.

The antigen binding domain may comprise SEQ ID NO: 79, or a variant which is at least 80% identical to SEQ ID NO: 79. Suitably, the variant may be at least 85, 90, 95, 97, 98 or 99% identical to SEQ ID NO: 79.

The antigen binding domain may comprise SEQ ID NO: 80, or a variant which is at least 80% identical to SEQ ID NO: 80. Suitably, the variant may be at least 85, 90, 95, 97, 98 or 99% identical to SEQ ID NO: 80.

The antigen binding domain may comprise SEQ ID NO: 81, or a variant which is at least 80% identical to SEQ ID NO: 81. Suitably, the variant may be at least 85, 90, 95, 97, 98 or 99% identical to SEQ ID NO: 81.

The antigen binding domain may comprise SEQ ID NO: 82, or a variant which is at least 80% identical to SEQ ID NO: 82. Suitably, the variant may be at least 85, 90, 95, 97, 98 or 99% identical to SEQ ID NO: 82.

The antigen binding domain may comprise SEQ ID NO: 83, or a variant which is at least 80% identical to SEQ ID NO: 83. Suitably, the variant may be at least 85, 90, 95, 97, 98 or 99% identical to SEQ ID NO: 83.

The antigen binding domain may comprise SEQ ID NO: 84, or a variant which is at least 80% identical to SEQ ID NO: 84. Suitably, the variant may be at least 85, 90, 95, 97, 98 or 99% identical to SEQ ID NO: 84.

The antigen binding domain may comprise SEQ ID NO: 85, or a variant which is at least 80% identical to SEQ ID NO: 85. Suitably, the variant may be at least 85, 90, 95, 97, 98 or 99% identical to SEQ ID NO: 85.

The antigen binding domain may comprise SEQ ID NO: 86, or a variant which is at least 80% identical to SEQ ID NO: 86. Suitably, the variant may be at least 85, 90, 95, 97, 98 or 99% identical to SEQ ID NO: 86.

Transmembrane Domain

The CAR may also comprise a transmembrane domain. The transmembrane domain may comprise the transmembrane sequence from any protein which has a transmembrane domain, including any of the type I, type II or type III transmembrane proteins. The transmembrane domain of the CAR may also comprise an artificial hydrophobic sequence. The transmembrane domains of the CAR may be selected so as not to dimerize. Additional transmembrane domains will be apparent to those of skill in the art. Examples of transmembrane (TM) regions used in CAR constructs are: 1) The CD28 TM region (Pule et al, Mol Ther, 2005, November; 12(5):933-41; Brentjens et al, CCR, 2007, Sep. 15; 13(18 Pt 1):5426-35; Casucci et al, Blood, 2013, Nov. 14; 122(20):3461-72.); 2) The OX40 TM region (Pule et al, Mol Ther, 2005, November; 12(5):933-41); 3) The 41BB TM region (Brentjens et al, CCR, 2007, Sep. 15; 13(18 Pt 1):5426-35); 4) The CD3 zeta TM region (Pule et al, Mol Ther, 2005, November; 12(5):933-41; Savoldo B, Blood, 2009, Jun. 18; 113(25):6392-402.); 5) The CD8a TM region (Maher et al, Nat Biotechnol, 2002, January; 20(1):70-5.; Imai C, Leukemia, 2004, April; 18(4):676-84; Brentjens et al, CCR, 2007, Sep. 15; 13(18 Pt 1):5426-35; Milone et al, Mol Ther, 2009, August; 17(8):1453-64.).

Suitably, the transmembrane domain may comprise the amino acid sequence shown as SEQ ID NO: 35, or a variant which is at least 80% identical to SEQ ID NO: 35

CD28 Transmembrane
SEQ ID NO: 35
FWVLVVVGGVLACYSLLVTVAFIIFWV

Suitably, the variant may be at least 85, 90, 95, 97, 98 or 99% identical to SEQ ID NO: 35.

Suitably, the CAR may comprise the CD8a transmembrane domain. Suitably, the transmembrane domain may comprise the amino acid sequence shown as SEQ ID NO: 87, or a variant which is at least 80% identical to SEQ ID NO: 87.

Illustrative CD8a TM domain (AA 183 to 203)
(SEQ ID NO: 87):
IYIWAPLAGTCGVLLLSLVIT

Suitably, the variant may be at least 85, 90, 95, 97, 98 or 99% identical to SEQ ID NO: 87

Suitably, the CAR may comprise the CD28 hinge and transmembrane sequence. Suitably, the hinge and transmembrane domain may comprise the amino acid sequence shown as SEQ ID NO: 36, or a variant which is at least 80% identical to SEQ ID NO: 36

CD28 transmembrane
SEQ ID NO: 36
IEVMYPPPYLDNEKSNGTIIHVKGKHLCPSPLFPGPSKPWFWLVVVGGVL
ACYSLLVTVAFIIFWV

Suitably, the variant may be at least 85, 90, 95, 97, 98 or 99% identical to SEQ ID NO: 36.

In one embodiment the transmembrane and intracellular signaling domain are both derived from CD28. In one embodiment the transmembrane and intracellular signaling domain comprise the sequence below:

Transmembrane and intracellular portion of the human CD28 (UNIPROT: P10747, CD28_HUMAN, position 153-220)

(SEQ ID NO: 37)
FWVLVVVGGVLACYSLLVTVAFIIFWVRSKRSRLLHSDYMNMTPRRPGPT
RKHYQPYAPPRDFAAYRS

In one embodiment the transmembrane and intracellular signaling domain comprises at least 85, 90, 95, 97, 98 or 99% identity to SEQ ID NO: 37.

In one embodiment the transmembrane domain of CD28 comprises the sequence

(SEQ ID NO: 38)
FWVLVVVGGVLACYSLLVTVAFIIFWV.

Suitably, the CAR may encode a tag—such as a c-Myc tag (EQKLISEEDL—SEQ ID NO: 39). Suitably the tag may be incorporated into the extracellular domain of the CAR, for example in the hinge region of the extracellular domain. An illustrative CD28 hinge/transmembrane domain with an integrated c-Myc tag is shown as SEQ ID NO: 40.

(SEQ ID NO: 40)
IEVEQKLISEEDLLDNEKSNGTIIHVKGKHLCPSPLFPGPSKPFWVLVVV
GGVLACYSLLVTVAFIIFWV.

Suitably, the CAR may comprise the CD8a hinge domain and the CD28 transmembrane domain. Suitably, the hinge and transmembrane domain may comprise the amino acid sequence shown as SEQ ID NO: 88, or a variant which is at least 80% identical to SEQ ID NO: 88.

Illustrative CD8α hinge domain and the CD28
transmembrane domain
(SEQ ID NO: 88):
TTTPAPRPPTPAPTIASQPLSLRPEACRPAAGGAVHTRGLDFACDFWVLV
VVGGVLACYSLLVTVAFIIFWV

Suitably, the variant may be at least 85, 90, 95, 97, 98 or 99% identical to SEQ ID NO: 88.

Suitably, the CAR may comprise the CD28 hinge domain and the CD8a transmembrane domain. Suitably, the hinge and transmembrane domain may comprise the amino acid sequence shown as SEQ ID NO: 89, or a variant which is at least 80% identical to SEQ ID NO: 89.

Illustrative CD28 hinge domain and the CD8α
transmembrane domain
(SEQ ID NO: 89):
IEVMYPPPYLDNEKSNGTIIHVKGKHLCPSPLFPGPSKPIYIWAPLAGTC
GVLLLSLVIT

Suitably, the variant may be at least 85, 90, 95, 97, 98 or 99% identical to SEQ ID NO: 89.

The CAR may further comprise a leader sequence which targets it to the endoplasmic reticulum pathway for expression on the cell surface. An illustrative leader sequence is MALPVTALLLPLALLLHAARP (SEQ ID NO: 41).

Illustrative CARs for use in the present invention are shown as SEQ ID NO: 42-44.

(CAR containing HLA-A2 scFV, c-Myc tag, CD28,
IL2RB-Y510, CD3 zeta endodomain)
SEQ ID NO: 42
MALPVTALLLPLALLLHAARPQVQLVQSGGGVVQPGGSLRVSCAASGVTL
SDYGMHWVRQAPGKGLEWMAFIRNDGSDKYYADSVKGRFTISRDNSKKTV
SLQMSSLRAEDTAVYYCAKNGESGPLDYWYFDLWGRGTLVTVSSGGGGSG
GGGSGGGGSDVVMTQSPSSLSASVGDRVTITCQASQDISNYLNWYQQKPG
KAPKLLIYDASNLETGVPSRFSGSGSGTDFTFTISSLQPEDIATYYCQQY
DNLPPTFGGGTKLTVLGAAAIEVEQKLISEEDLLDNEKSNGTIIHVKGKH
LCPSPLFPGPSKPFWVLVVVGGVLACYSLLVTVAFIIFWVRSKRSRLLHS
DYMNMTPRRPGPTRKHYQPYAPPRDFAAYRSNCRNTGPWLKKVLKCNTPD
PSKFFSQLSSEHGGDVQKWLSSPFPSSSFSPGGLAPEISPLEVLERDKVT
QLLPLNTDAYLSLQELQGQDPTHLVRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPRETRGGGATMVSKG
EELFTGVVPILVELDGDVNGHKFSVSGEGEGDATYGKLTLKFICTTGKLP
VPWPTLVTTLTYGVQCFSRYPDHMKQHDFFKSAMPEGYVQERTIFFKDDG
NYKTRAEVKFEGDTLVNRIELKGIDFKEDGNILGHKLEYNYNSHNVYIMA
DKQKNGIKVNFKIRHNIEDGSVQLADHYQQNTPIGDGPVLLPDNHYLSTQ
SKLSKDPNEKRDHMVLLEFVTAAGITLGMDELYK
(CAR containing HLA-A2 scFV, c-Myc tag, CD28,
IL2RG-T52, IL2RB-Y510, CD3 zeta endodomain)
SEQ ID NO: 43
MALPVTALLLPLALLLHAARPQVQLVQSGGGVVQPGGSLRVSCAASGVTL
SDYGMHWVRQAPGKGLEWMAFIRNDGSDKYYADSVKGRFTISRDNSKKTV
SLQMSSLRAEDTAVYYCAKNGESGPLDYWYFDLWGRGTLVTVSSGGGGSG
GGGSGGGGSDVVMTQSPSSLSASVGDRVTITCQASQDISNYLNWYQQKPG
KAPKLLIYDASNLETGVPSRFSGSGSGTDFTFTISSLQPEDIATYYCQQY
DNLPPTFGGGTKLTVLGAAAIEVEQKLISEEDLLDNEKSNGTIIHVKGKH
LCPSPLFPGPSKPFWVLVVVGGVLACYSLLVTVAFIIFWVRSKRSRLLHS
DYMNMTPRRPGPTRKHYQPYAPPRDFAAYRSERTMPRIPTLKNLEDLVTE
YHGNFSAWSGVSKGLAESLQPDYSERLCLVSEINCRNTGPWLKKVLKCNT
PDPSKFFSQLSSEHGGDVQKWLSSPFPSSSFSPGGLAPEISPLEVLERDK
VTQLLPLNTDAYLSLQELQGQDPTHLVRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPR
(CAR containing HLA-A2 scFV, c-Myc tag, CD28,
IL2RG-T52, IL7RA-2Y, CD3 zeta endodomain)
SEQ ID NO: 44
MALPVTALLLPLALLLHAARPQVQLVQSGGGVVQPGGSLRVSCAASGVTL
SDYGMHWVRQAPGKGLEWMAFIRNDGSDKYYADSVKGRFTISRDNSKKTV
SLQMSSLRAEDTAVYYCAKNGESGPLDYWYFDLWGRGTLVTVSSGGGGSG
GGGSGGGGSDVVMTQSPSSLSASVGDRVTITCQASQDISNYLNWYQQKPG
KAPKLLIYDASNLETGVPSRFSGSGSGTDFTFTISSLQPEDIATYYCQQY
DNLPPTFGGGTKLTVLGAAAIEVEQKLISEEDLLDNEKSNGTIIHVKGKH
LCPSPLFPGPSKPFWVLVVVGGVLACYSLLVTVAFIIFWVRSKRSRLLHS
DYMNMTPRRPGPTRKHYQPYAPPRDFAAYRSERTMPRIPTLKNLEDLVTE
YHGNFSAWSGVSKGLAESLQPDYSERLCLVSEIKKRIKPIVWPSLPDHKK
TLEHLCKKPRKNLNVSFNPESFLDCQIHRVDDIQARDEVEGFLQDTFPQQ
PILTSLGSNQEEAYVTMSSFYQNQRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPRGSGATNFSLLKQAG
DVEENPG

The CAR may comprise a sequence which is at least 85, 90, 95, 97, 98 or 99% identity to any one of SEQ ID NO: 42-44.

Pharmaceutical Composition

There is also provided a pharmaceutical composition comprising an engineered Treg, or CAR, of the invention.

A pharmaceutical composition is a composition that comprises or consists of a therapeutically effective amount of a pharmaceutically active agent i.e. the Treg. It preferably includes a pharmaceutically acceptable carrier, diluent or excipient (including combinations thereof). Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” is included that the formulation is sterile and pyrogen free. The carrier, diluent, and/or excipient must be “acceptable” in the sense of being compatible with the Treg and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The Tregs or pharmaceutical compositions according to the present invention may be administered in a manner appropriate for treating and/or preventing the disease described herein. The quantity and frequency of administration will be determined by such factors as the condition of the subject, and the type and severity of the subjects's disease, although appropriate dosages may be determined by clinical trials. The pharmaceutical composition may be formulated accordingly.

The Treg or pharmaceutical composition as described herein can be administered parenterally, for example, intravenously, or they may be administered by infusion techniques. The Treg or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The pharmaceutical compositions may comprise Tregs of the invention in infusion media, for example sterile isotonic solution. The pharmaceutical composition may be enclosed in ampoules, disposable syringes or multiple dose vials made of glass or plastic.

The Treg or pharmaceutical composition may be administered in a single or in multiple doses. Particularly, the Treg or pharmaceutical composition may be administered in a single, one off dose. The pharmaceutical composition may be formulated accordingly.

The pharmaceutical composition may further comprise one or more active agents.

The pharmaceutical composition may further comprise one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4Ig), and/or drugs inhibiting specific cytokines (IL-6, IL-17, TNFalpha, IL18).

Depending upon the disease and subject to be treated, as well as the route of administration, the Treg or pharmaceutical composition may be administered at varying doses (e.g. measured in cells/kg or cells/subject). The physician in any event will determine the actual dosage which will be most suitable for any individual subject and it will vary with the age, weight and response of the particular subject. Typically, however, for Tregs of the invention, doses of 5×10⁷ to 3×10⁹ cells, or 10⁸ to 2×10⁹ cells per subject may be administered.

The Treg may be appropriately modified for use in a pharmaceutical composition. For example, Tregs may be cryopreserved and thawed at an appropriate time, before being infused into a subject.

The invention further includes the use of kits comprising the Treg and/or pharmaceutical composition of the present invention. Preferably said kits are for use in the methods and uses as described herein, e.g., the therapeutic methods as described herein. Preferably said kits comprise instructions for use of the kit components.

Method of Treatment

The present invention provides a method for inducing tolerance to a transplant; treating and/or preventing cellular and/or humoral transplant rejection; treating and/or preventing graft-versus-host disease (GvHD), an autoimmune or allergic disease; or to promote tissue repair and/or tissue regeneration; or to ameliorate chronic inflammation secondary to metabolic disorders which comprises the step of administering an engineered Treg or a pharmaceutical composition of the invention to a subject.

As used herein, “inducing tolerance to a transplant” refers to inducing tolerance to a transplanted organ in a recipient. In other words, inducing tolerance to a transplant means to reduce the level of a recipient's immune response to a donor transplant organ. Inducing tolerance to a transplanted organ may reduce the amount of immunosuppressive drugs that a transplant recipient requires, or may enable the discontinuation of immunosuppressive drugs.

For example, the engineered Tregs may be administered to a subject with a disease in order to lessen, reduce, or improve at least one symptom of disease such as jaundice, dark urine, itching, abdominal swelling or tenderness, fatigue, nausea or vomiting, and/or loss of appetite. The at least one symptom may be lessened, reduced, or improved by at least 10%, at least 20%, at least 30%, at least 40%, or at least 50%, or the at least one symptom may be completely alleviated.

The engineered Tregs may be administered to a subject with a disease in order to slow down, reduce, or block the progression of the disease. The progression of the disease may be slowed down, reduced, or blocked by at least 10%, at least 20%, at least 30%, at least 40%, or at least 50% compared to a subject in which the engineered Tregs are not administered, or progression of the disease may be completely stopped.

In one embodiment, the subject is a transplant recipient undergoing immunosuppression therapy.

Suitably, the subject is a mammal. Suitably, the subject is a human.

The transplant may be selected from a liver, kidney, heart, lung, pancreas, intestine, stomach, bone marrow, vascularized composite tissue graft, and skin transplant.

Suitably, the CAR may comprise an antigen binding domain which is capable of specifically binding to a HLA antigen that is present in the graft (transplant) donor but not in the graft (transplant) recipient.

Suitably, the transplant is a liver transplant. In embodiments where the transplant is a liver transplant, the antigen may be a HLA antigen present in the transplanted liver but not in the patient, a liver-specific antigen such as NTCP, or an antigen whose expression is up-regulated during rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

Suitably, the antigen may be HLA-A2.

The present invention further provides a method for treating and/or preventing graft-versus-host disease (GvHD), an autoimmune or allergic disease; or to promote tissue repair and/or tissue regeneration; or to ameliorate chronic inflammation secondary to metabolic disorders.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

Suitably, treating and/or preventing cellular and/or humoral transplant rejection may refer to administering an effective amount of a Treg of the invention such that the amount of immunosuppressive drugs that a transplant recipient requires is reduced, or may enable the discontinuation of immunosuppressive drugs.

Preventing a disease relates to the prophylactic use of the cells of the present invention. In this respect, the cells may be administered to a subject who has not yet contracted the disease and/or who is not showing any symptoms of the disease to prevent the disease or to reduce or prevent development of at least one symptom associated with the disease. The subject may have a predisposition for, or be thought to be at risk of developing, the disease.

The autoimmune or allergic disease may be selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); multiple sclerosis and juvenile onset diabetes.

Suitably, the therapeutic methods of the invention may comprise the step of administering an engineered Treg according to the invention, or obtainable (e.g. obtained) by a method according to the present invention, or a polynucleotide or a vector as defined herein (for example in a pharmaceutical composition as described above) to a subject.

Suitably, the present methods for treating and/or preventing a disease may comprise administering an engineered Treg according to the present invention (for example in a pharmaceutical composition as described above) to a subject.

The method may involve the steps of:

(i) isolating a cell-containing sample or providing a cell-containing sample;

(ii) introducing a polynucleotide or a vector as defined herein to the cell; and

(iii) administering the cells from (ii) to a subject.

Suitably, the cell is a Treg as defined herein.

Suitably, an enriched Treg population may be isolated and/or generated from the cell containing sample prior to, and/or after, step (ii) of the method.

For example, isolation and/or generation may be performed prior to and/or after step (ii) to isolate and/or generate an enriched Treg sample. Enrichment may be performed after step (ii) to enrich for cells and/or Tregs comprising the CAR, the polynucleotide, and/or the vector of the present invention.

Suitably, the polynucleotide or vector may be introduced by transduction. Suitably, the polynucleotide or vector may be introduced by transfection.

Suitably, the cell may be autologous. Suitably, the cell may be allogenic.

Suitably, the engineered Treg may be administered is combination with one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4Ig), and/or drugs inhibiting specific cytokines (IL-6, IL-17, TNFalpha, IL18). The engineered Treg may be administered simultaneously with or sequentially with (i.e. prior to or after) the one or more other therapeutic agents.

Suitably the subject is a mammal. Suitably the subject is a human.

Tregs may be activated and/or expanded prior to, or after, the introduction of a polynucleotide encoding the CAR as described herein, for example by treatment with an anti-CD3 monoclonal antibody or both anti-CD3 and anti-CD28 monoclonal antibodies.

The Tregs may also be expanded in the presence of anti-CD3 and anti-CD28 monoclonal antibodies in combination with IL-2. Suitably, IL-2 may be substituted with IL-15. Other components which may be used in a Treg expansion protocol include, but are not limited to rapamycin, all-trans retinoic acid (ATRA) and TGFβ.

As used herein “activated” means that a Treg or population of Tregs has been stimulated, causing the Treg(s) to proliferate. As used herein “expanded” means that a Treg or population of Tregs has been induced to proliferate. The expansion of a population of Tregs may be measured for example by counting the number of Tregs present in a population. The phenotype of the Tregs may be determined by methods known in the art such as flow cytometry.

The Tregs may be washed after each step of the method, in particular after expansion.

The population of engineered Treg cells according to the present invention may be further enriched by any method known to those of skill in the art, for example by FACS or magnetic bead sorting.

The steps of the method of production may be performed in a closed and sterile cell culture system.

Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that the skilled person may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

The present polynucleotide may further comprise a nucleic acid sequence encoding a selectable marker. Suitably selectable markers are well known in the art and include, but are not limited to, fluorescent proteins—such as GFP. Suitably, the selectable marker may be a fluorescent protein, for example GFP, YFP, RFP, tdTomato, dsRed, or variants thereof. In some embodiments the fluorescent protein is GFP or a GFP variant. The nucleic acid sequence encoding a selectable marker may be provided in combination with a nucleic acid sequence encoding the present CAR in the form of a nucleic acid construct. Such a nucleic acid construct may be provided in a vector.

Suitably, the selectable marker/reporter domain may be a luciferase-based reporter, a PET reporter (e.g. Sodium Iodide Symporter (NIS)), or a membrane protein (e.g. CD34, low-affinity nerve growth factor receptor (LNGFR)).

The nucleic acid sequences encoding the CAR and the selectable marker may be separated by a co-expression site which enables expression of each polypeptide as a discrete entity. Suitable co-expression sites are known in the art and include, for example, internal ribosome entry sites (IRES) and self-cleaving peptides.

Further suitable co-expression sites/sequences include self-cleaving or cleavage domains. Such sequences may either auto-cleave during protein production or may be cleaved by common enzymes present in the cell. Accordingly, inclusion of such self-cleaving or cleavage domains in the polypeptide sequence enables a first and a second polypeptide to be expressed as a single polypeptide, which is subsequently cleaved to provide discrete, separated functional polypeptides.

Suitable self-cleaving or cleavage domains include, but are not limited to, those shown as SEQ ID NO: 46-51.

P2A peptide-cleavage domain:
(SEQ ID NO: 46)
GSGATNFSLLKQAGDVEENPGP
T2A peptide-cleavage domain:
(SEQ ID NO: 47)
GSGEGRGSLLTCGDVEENPGP
E2A peptide-cleavage domain:
(SEQ ID NO: 48)
GSGQCTNYALLKLAGDVESNPGP
F2A peptide-cleavage domain:
(SEQ ID NO: 49)
GSGVKQTLNFDLLKLAGDVESNPGP
Furin site-cleavage domain:
(SEQ ID NO: 50)
RXXR (preferentially: RRKR-SEQ ID NO: 51).

The use of a selectable marker is advantageous as it allows Treg in which a polynucleotide or vector of the present invention has been successfully introduced (such that the encoded CAR is expressed) to be selected and isolated from a starting cell population using common methods, e.g. flow cytometry.

Codon Optimisation

The polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Vectors

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest.

The vectors used in the invention may be, for example, plasmid, mRNA or virus vectors and may include a promoter for the expression of a polynucleotide and optionally a regulator of the promoter.

Vectors comprising polynucleotides of the invention may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Non-viral delivery systems can include liposomal or amphipathic cell penetrating peptides, preferably complexed with a polynucleotide of the invention.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

Multiple vectors, e.g. encoding different CARs or the invention, or encoding a CAR of the invention and a further polypeptide could be used for transduction/transfection.

Method of Making a Cell

Engineered Tregs of the present invention may be generated by introducing DNA or RNA coding for the CAR as defined herein, by one of many means including transduction with a viral vector, transfection with DNA or RNA.

The cell of the invention may be made by: introducing to a cell (e.g. by transduction or transfection) the polynucleotide or vector as defined herein.

Suitably, the cell may be from a sample isolated from a subject.

The engineered Treg of the present invention may be generated by a method comprising the following steps:
(i) isolation of a cell-containing sample from a subject or provision of a cell-containing sample; and
(ii) transduction or transfection of the cell-containing sample with a polynucleotide, a nucleic acid, or a vector encoding the CAR of the invention, to provide a population of engineered cells.

Suitably, a Treg-enriched sample may be isolated from, enriched, and/or generated from the cell-containing sample prior to and/or after step (ii) of the method. For example, isolation, enrichment and/or generation of Tregs may be performed prior to and/or after step (ii) to isolate, enrich or generate a Treg-enriched sample. Isolation and/or enrichment may be performed after step (ii) to enrich for cells and/or Tregs comprising the CAR, the polynucleotide, and/or the vector of the present invention.

A Treg-enriched sample may be isolated or enriched by any method known to those of skill in the art, for example by FACS and/or magnetic bead sorting. A Treg-enriched sample may be generated from the cell-containing sample by any method known to those of skill in the art, for example from Tcon cells by introducing DNA or RNA coding for FOXP3 and/or from ex-vivo differentiation of inducible progenitor cells or embryonic progenitor cells.

Suitably, the cell is a Treg as defined herein.

Suitably, the engineered Treg of the present invention may be generated by a method comprising the following steps:

(i) isolation of a Treg-enriched sample from a subject or provision of a Treg-enriched sample; and

(ii) transduction or transfection of the Treg-enriched sample with a polynucleotide, a nucleic acid, or a vector encoding the CAR of the invention, to provide a population of engineered Treg cells according to the present invention.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

The invention will now be further described by way of Examples, which are meant to serve to assist one of ordinary skill in the art in carrying out the invention and are not intended in any way to limit the scope of the invention.

### EXAMPLES

### Example 1—Generation of Anti-HLA.A2 IL2R CAR-Tregs

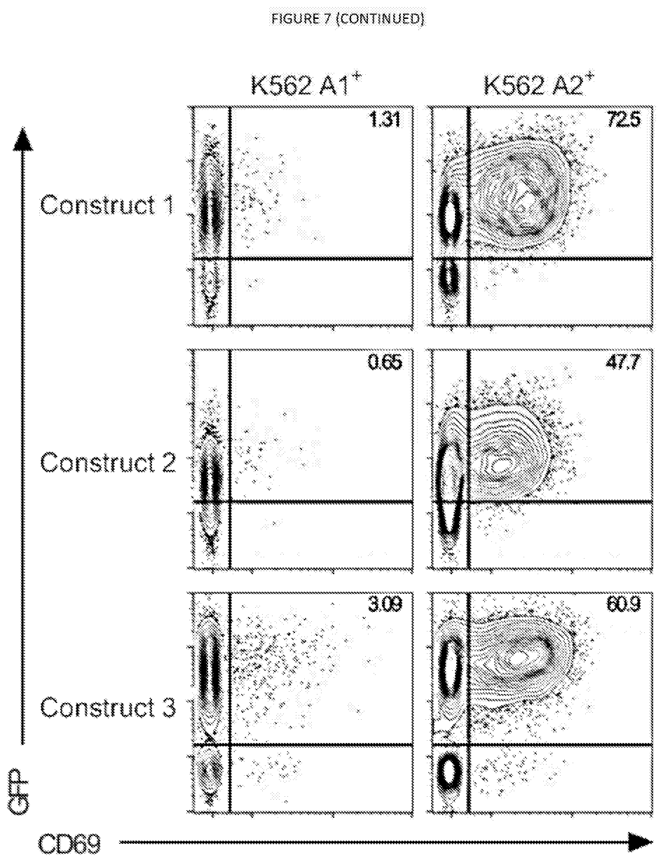

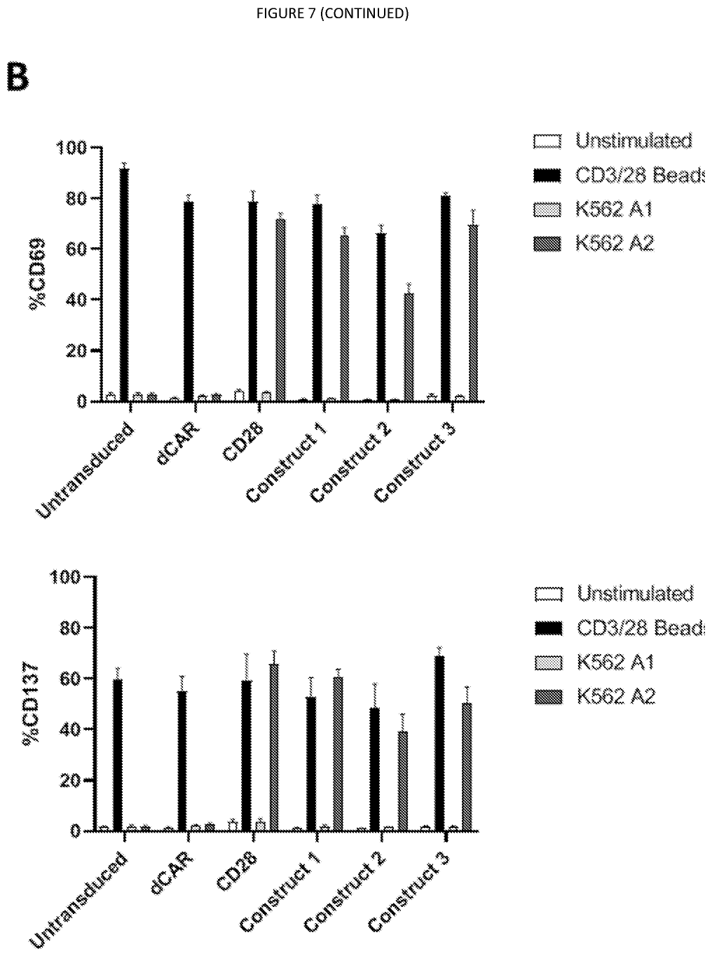

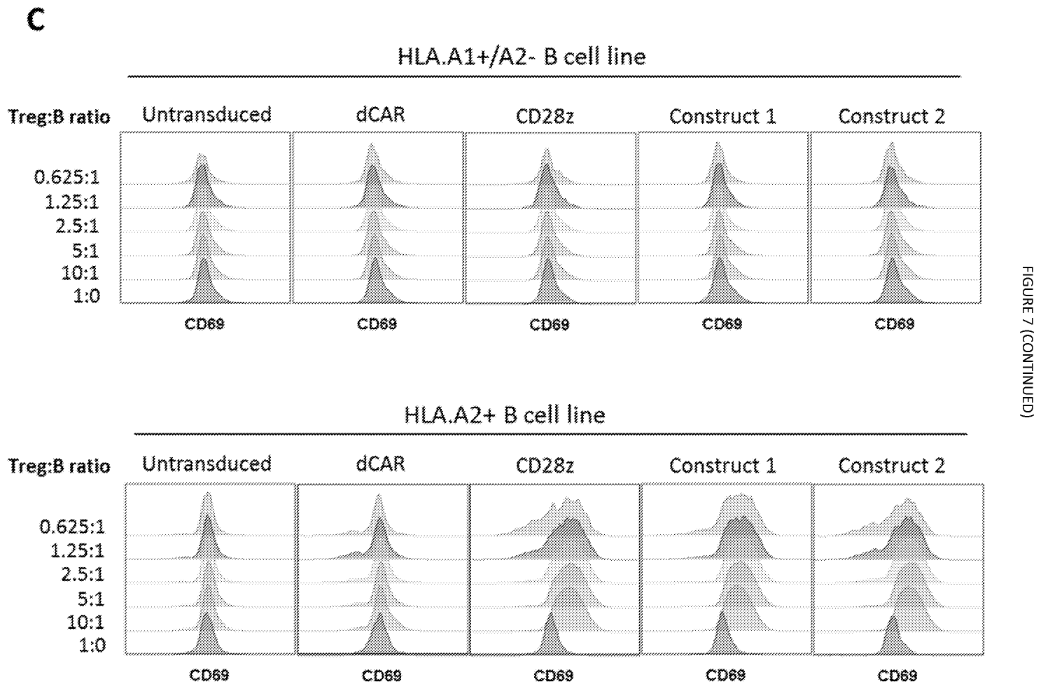

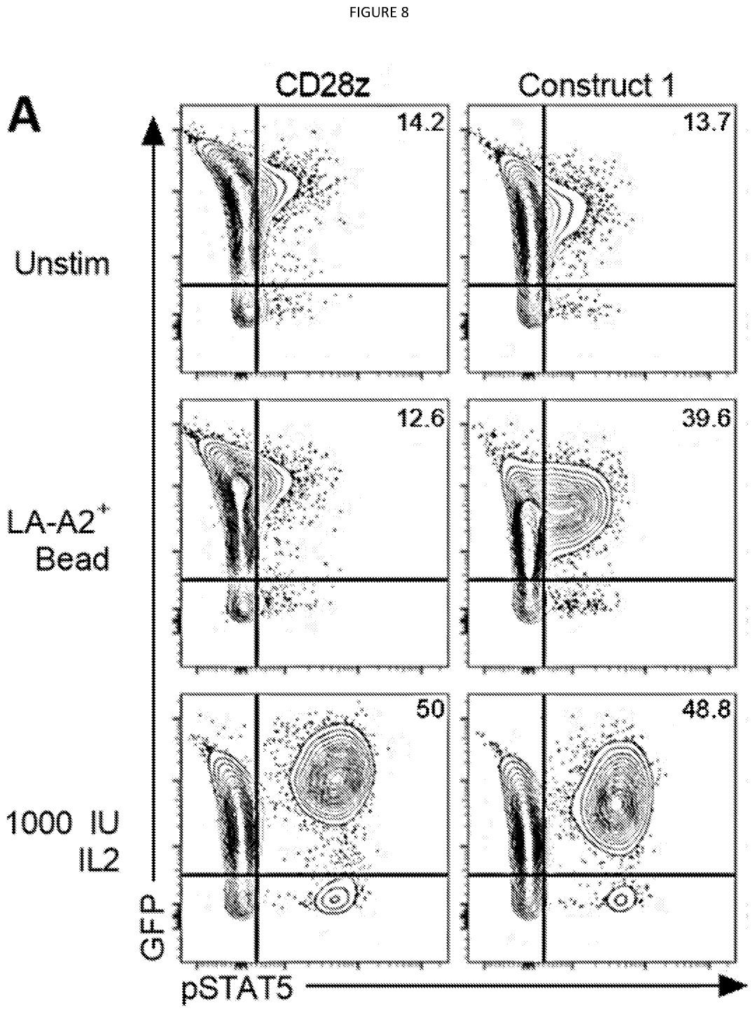

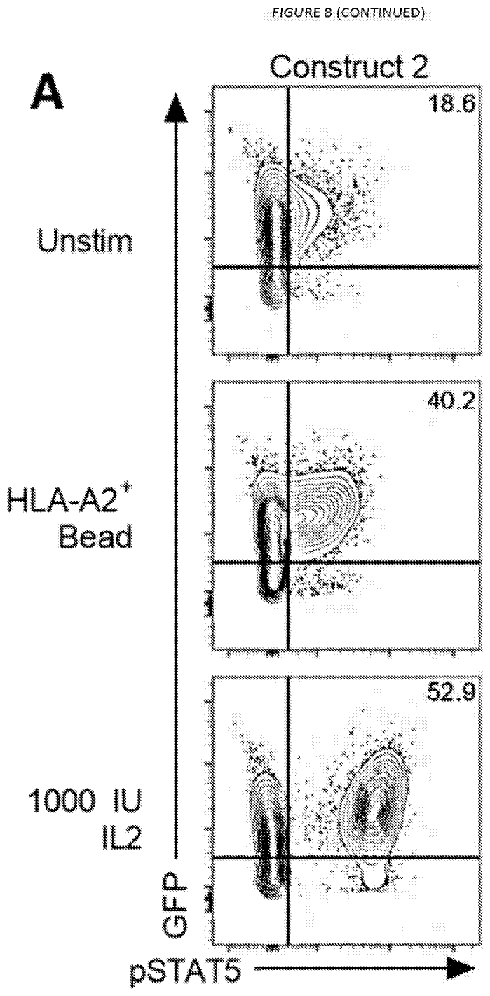

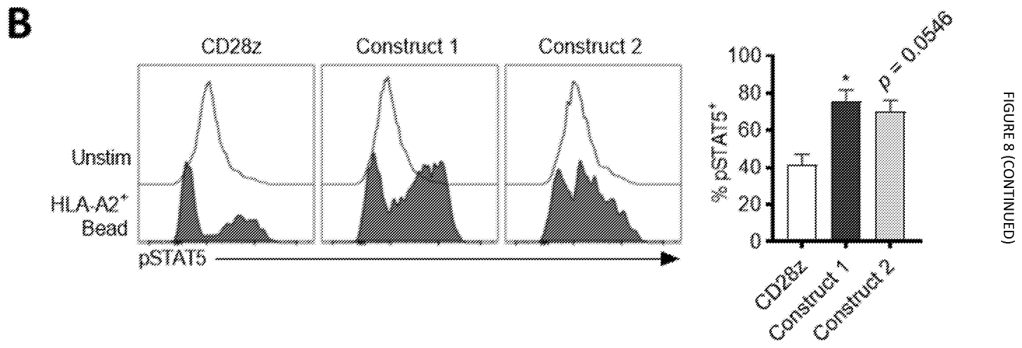

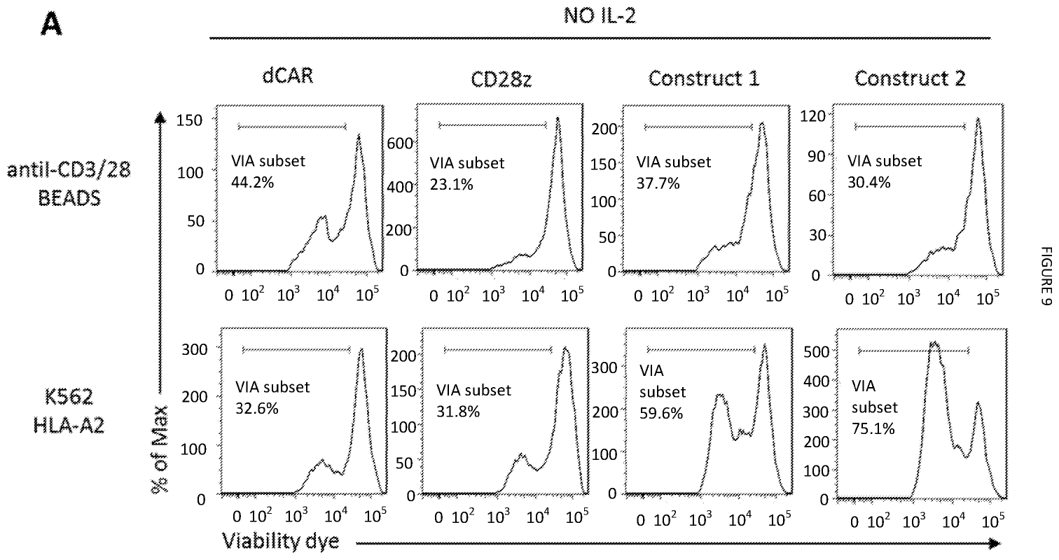

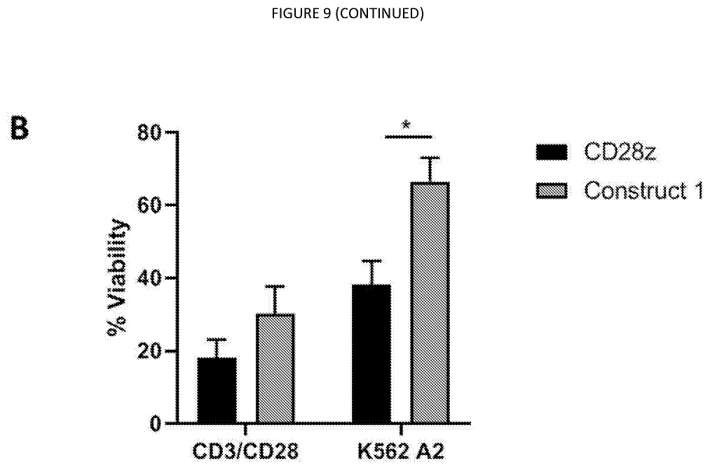

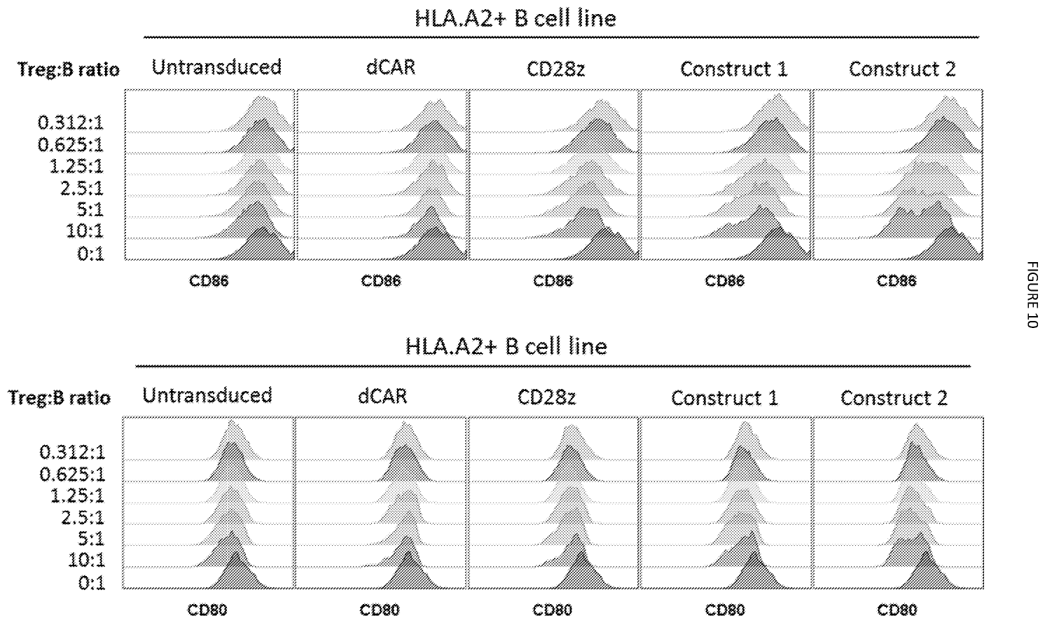

CD4+CD25hiCD127low cells were isolated and activated with anti-CD3/CD28 beads. Three days after activation Tregs were transduced with lentivirus containing the HLA.A2-CAR (shown in FIG. 2) and GFP reporter gene. Cellular expansion of total Tregs after polyclonal activation showed no significant differences between untransduced or transduced Treg (FIG. 3).

### Example 2—Quantification of Transduction Efficacy of Anti-HLA.A2 IL2R Constructs Over Time

GFP expression was analysed on Tregs untransduced and transduced with CAR constructs at different time points after cell activation.

Frequency of GFP+ cells was analysed to evaluate the transduction efficacy and the expression persistence of the different constructs over the Treg expansion period. Tregs containing dCAR, CD28z, Construct 1, 2 and 3 showed similar expression frequencies after transduction. The percentages of GFP+ cells among whole Tregs were maintained during polyclonal cellular expansion (FIG. 4).

### Example 3—Quantification of Cell Surface Expression of Anti-HLA.A2 IL2R CAR Constructs on Transduced Tregs

Membrane expression of CAR construct on untransduced and transduced Tregs was analysed by PE-conjugated HLA-A*0201/CINGVCWTV dextramers (Immudex, Copenhagen, Denmark). The frequency of Tregs expressing the CAR protein in the cell surface (HLA-A2 dextramer+) was similar between all the constructs (FIG. 5).

### Example 4—Phenotypic Characterization of CAR Tregs after Polyclonal Cell Expansion

Tregs were cultured and expanded for 15 days in the presence of anti-CD3/CD28 activation beads and IL-2. Treg related markers FOXP3, HELIOS, CTLA4 and TIGIT were analysed by FACS on untransduced and transduced Tregs to assess phenotypic lineage stability on day 15 of culture.

Untransduced and CAR-transduced showed similar expression levels of proteins associated with Treg lineage and function after polyclonal expansion (FIG. 6).

### Example 5—Evaluation of the Antigen-Specificity of Anti-HLA.A2 IL2R CAR Tregs

Untransduced and transduced Tregs were cultured for 18 hours in the presence of different stimulus. CD69 and CD137 activation markers were analysed to assess specific and unspecific cell activation.

Transduced Tregs with the CD28z, Construct 1, 2 and 3 CARs showed similar specificity for HLA-A2 molecules based on the expression of T cell activation markers. The expression of CD69 and CD137 was not increase on inactivated cells or after the culture with HLA-A1 expressing cells. The dCAR construct showed no activation due to the lack of signaling endodomains (FIG. 7).

### Example 6—STAT5 Phosphorylation Analysis as an Indicator of IL2R CAR Signaling

Transduced CAR Tregs were rested overnight in culture media without IL2. STAT5 phosphorylation of Tregs was assessed by FACS analysis 10 and 120 minutes after culture with media alone, 1000 IU/ml IL-2 or in the presence of HLA.A2-Ig based artificial APCs (produced following the protocol described at DOI: 10.3791/2801).

The integration of the IL2R endodomains into the CAR construct showed efficient phosphorylation of STAT5 after the CAR activation by HLA-A2 molecules. No significant increase of pSTAT5 was detected on CAR-Tregs without the IL2R endodomains after culture with HLA-A2 beads (FIG. 8).

### Example 7—Evaluation of Treg Survival after Unspecific and HLA.A2 Specific Activation in the Absence of IL-2

CAR transduced Tregs with different constructs were cultured with anti-CD3/28 activation beads and K562.A2 expression cells without the presence of IL-2. Cell survival was assessed 7 days after activation by FACS analysis.

Tregs expressing a CAR construct containing the IL2R endodomain showed increased cell viability compared to the reference CD28z after the cell culture with HLA-A2 expression cells. This differences were not observed after polyclonal activation of the Tregs demonstrating that the effect is dependent on CAR signalling (FIG. 9).

### Example 8—Treg Suppression Potency Test: Evaluate the Immunoregulatory Function of Tregs by Analysing the Modulation of Co-Stimulatory Molecules on B Cells

B cell expression of CD80 and CD86 after co-culture with Tregs was analysed to evaluate the capacity of Tregs to reduce the expression of co-stimulatory molecules on antigen presenting cells.

Tregs expressing the CD28z, Construct 1 and Construct 2 CARs showed increased suppressive function compared to untransduce or dCAR expressing Tregs. CD80 and CD86 expression on B cells is only downregulated after culture with Tregs that signal through the CAR molecule (FIG. 10).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. An engineered regulatory T cell (Treg) comprising a chimeric antigen receptor (CAR) for use in induction of tolerance to a transplant; treating and/or preventing graft-versus-host disease (GvHD), an autoimmune or allergic disease; to promote tissue repair and/or tissue regeneration; or to ameliorate chronic inflammation secondary to metabolic disorders; wherein the CAR comprises an endodomain which comprises a STAT5 association motif and a JAK1- and/or a JAK2-binding motif.

2. An engineered Treg for use according to claim 1 wherein the Treg is a Foxp3+ Treg.

3. An engineered Treg for use according to claim 1 or claim 2 wherein the CAR endodomain does not comprise a STAT3 association motif.

4. An engineered Treg for use according to claim 1 or claim 2 wherein the CAR endodomain does not comprise the amino acid sequence YXXQ (SEQ ID NO: 52).

5. An engineered Treg for use according to any preceding claim wherein the CAR endodomain comprises two or more STAT5 association motifs.

6. An engineered Treg for use according to any preceding claim wherein the one or more STAT5 association motifs is from an interleukin receptor (IL) receptor endodomain.

7. An engineered Treg for use according to any of claims 1 to 6 wherein the one or more STAT5 association motifs is from IL2Rβ, IL7Rα, IL-3Rβ (CSF2RB), IL-9R, IL-17Rβ, erythropoietin receptor, thrombopoietin receptor, growth hormone receptor and prolactin receptor.

8. An engineered Treg for use according to any preceding claim wherein the STAT5 association motif comprises the amino acid motif YXXF/L (SEQ ID NO: 8); wherein X is any amino acid.

9. An engineered Treg for use according to any preceding claim wherein the STAT5 association motif comprises one or more of the amino acid motifs YCTF (SEQ ID NO: 9), YFFF (SEQ ID NO: 10), YLSL (SEQ ID NO: 11), and/or YLSLQ (SEQ ID NO: 12).

10. An engineered Treg for use according to claim 9 wherein the STAT5 association motif comprises the amino acid motif YLSLQ (SEQ ID NO: 12).

11. An engineered Treg for use according to claim 10 wherein the endodomain comprises a first STAT5 association motif comprising the amino acid motif YLSLQ (SEQ ID NO: 12) and a second STAT5 association motif comprising the amino acid motif YCTF (SEQ ID NO: 9) or YFFF (SEQ ID NO: 10).

12. An engineered Treg for use according to claim 11 wherein the endodomain comprises the following STAT5 association motifs: YLSLQ (SEQ ID NO: 12), YCTF (SEQ ID NO: 9) and YFFF (SEQ ID NO: 10).

13. An engineered Treg for use according to any preceding claim wherein the JAK-binding motif is a JAK-1 binding motif.

14. An engineered Treg for use according to claim 13 wherein the JAK1-binding motif is from an interleukin receptor (IL) receptor endodomain.

15. An engineered Treg for use according to any preceding claim wherein the JAK1-binding motif comprises an amino acid motif shown as any one of SEQ ID NO: 13-19 or a variant which has at least 80% identity to SEQ ID NO: 13-19.

16. An engineered Treg for use according to claim 15 wherein the JAK1-binding motif is the amino acid motif shown as SEQ ID NO: 13; or a variant which has at least 80% identity to SEQ ID NO: 13.

17. An engineered Treg for use according to any preceding claim wherein the CAR endodomain comprises an IL2Rβ endodomain shown as SEQ ID NO: 1; or a variant which has at least 80% sequence identity to SEQ ID NO: 1.

18. An engineered Treg for use according to any of claims 1-17 wherein the CAR endodomain comprises a truncated IL2Rβ endodomain shown as any one of SEQ ID NO: 23 or 24; or a variant of SEQ ID NO: 23 or 24 which has at least 80% sequence identity thereto.

19. An engineered Treg for use according to any preceding claim wherein the CAR endodomain further comprises a JAK3-binding motif.

20. An engineered Treg for use according to claim 19 wherein the JAK3-binding motif comprises SEQ ID NO: 25 or 26 or a variant which has at least 80% sequence identity to SEQ ID NO: 25 or 26.

21. An engineered Treg for use according to claim 19 or 20 wherein the CAR endodomain comprises SEQ ID NO: 45 or 53; or a variant which has at least 80% sequence identity to SEQ ID NO: 45 or 53.

22. A pharmaceutical composition comprising an engineered Treg as defined in any of claims 1 to 21 for use in induction of tolerance to a transplant; treating and/or preventing graft-versus-host disease (GvHD), an autoimmune or allergic disease; to promote tissue repair and/or tissue regeneration; or to ameliorate chronic inflammation secondary to metabolic disorders.

23. A method of inducing tolerance to a transplant; treating and/or preventing graft-versus-host disease (GvHD), an autoimmune or allergic disease; or to promote tissue repair and/or tissue regeneration; or to ameliorate chronic inflammation secondary to metabolic disorders which comprises the step of administering an engineered Treg as defined in any of claims 1 to 20 or a pharmaceutical composition comprising an engineered Treg as defined in any of claims 1 to 20 to a subject.

24. A method according to claim 23 which comprises the following steps:
(i) isolation or provision of a Treg-enriched cell sample from a subject;
(ii) transduction or transfection of the Treg cells with: a polynucleotide; a nucleic acid construct; or a vector encoding a CAR as defined in any of claims 1 to 20; and
(iii) administering the Treg cells from (ii) to the subject.

25. Use of an engineered Treg as defined in any of claims 1 to 19 in the manufacture of a medicament for inducing tolerance to a transplant; treating and/or preventing cellular and/or humoral transplant rejection; treating and/or preventing graft-versus-host disease (GvHD), an autoimmune or allergic disease; or to promote tissue repair and/or tissue regeneration; or to ameliorate chronic inflammation secondary to metabolic disorders.

26. An engineered Treg or pharmaceutical composition for use according to any of claims 1 to 22; a method according to claim 23 or 24; or the use according to claim 25 wherein the subject is a transplant recipient undergoing immunosuppression therapy.

27. An engineered Treg or pharmaceutical composition for use; a method according to; or the use according to claim 26 wherein the transplant is selected from a liver, kidney, heart, lung, pancreas, intestine, stomach, bone marrow, vascularized composite tissue graft, and skin transplant.

28. An engineered Treg or pharmaceutical composition for use; a method; or the use according to claim 27 wherein the transplant is a liver transplant.

29. An engineered Treg or pharmaceutical composition for use; a method or the use according to claim 28 wherein the CAR comprises an antigen binding domain which is capable of specifically binding to an antigen selected from: a HLA antigen present in the transplanted liver but not in the recipient, a liver-specific antigen such as NTCP, or an antigen whose expression is up-regulated during rejection or tissue inflammation such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

30. An engineered Treg or pharmaceutical composition for use; a method or the use according to claim 29 wherein the CAR comprises an antigen binding domain which is capable of specifically binding to a HLA antigen that is present in the graft donor but not in the graft recipient.

31. An engineered Treg or pharmaceutical composition for use; a method or the use according to claim 30 wherein the antigen is HLA-A2.

32. An engineered Treg or pharmaceutical composition for use; a method or the use according to claim 31 wherein the CAR comprises an antigen binding domain comprises SEQ ID NO: 34 or a variant of SEQ ID NO: 34 with at least 80% identity thereto.

33. An engineered Treg or pharmaceutical composition for use; a method or the use according to and of claims 1 to 25 wherein the autoimmune or allergic disease is selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); multiple sclerosis and juvenile onset diabetes.

34. A chimeric antigen receptor (CAR) comprising an endodomain which comprises a STAT5 association motif and a JAK1- and/or a JAK2-binding motif but does not comprise a STAT3 association motif.

35. A CAR comprising an endodomain which comprises a STAT5 association motif and a JAK1- and/or a JAK2-binding motif but does not comprise the amino acid sequence YXXQ (SEQ ID NO: 52).

36. A chimeric antigen receptor (CAR) comprising an endodomain which comprises a STAT5 association motif, a JAK1- and/or a JAK2-binding motif, and a JAK3-binding motif.

37. A CAR according to claim 36 wherein the endodomain does not comprise a STAT3 association motif.

38. A CAR according to claim 36 wherein the endodomain does not comprise the amino acid sequence YXXQ (SEQ ID NO: 52).

39. A CAR according to any of claims 36 to 38 wherein the JAK3-binding motif comprises SEQ ID NO: 25 or 26 or a variant which has at least 80% sequence identity to SEQ ID NO: 25 or 26.

40. A CAR according to any of claims 36 to 39 wherein the CAR endodomain comprises SEQ ID NO: 45 or 53; or a variant which has at least 80% sequence identity to SEQ ID NO: 45 or 53.

41. A polynucleotide encoding a CAR according to any of claims 34 to 40.

42. An engineered Foxp3+ Treg comprising a chimeric antigen receptor (CAR) according to any of claims 34 to 40 or a polynucleotide according to claim 41.

43. A method of producing an engineered Treg according to claim 42, comprising the following steps:
(i) isolation of a cell-containing sample from a subject or provision of a cell-containing sample; and
(ii) transduction or transfection of the cell-containing sample with a polynucleotide, a nucleic acid, or a vector encoding the CAR, to provide a population of engineered cells;
wherein the cell-containing sample comprises Tregs and/or Tregs are enriched and/or generated from the cell-containing sample prior to or after step (ii).
